(19) United States
(12) Patent Application Publication
(10) Pub. No.: US 2023/0265516 A1
(43) Pub. Date: Aug. 24, 2023

(54) SINGLE NUCLEOTIDE POLYMORPHISMS AND TREATMENT OF INFLAMMATORY CONDITIONS

(71) Applicant: UNIVERSITY OF ARIZONA, ARIZONA BOARD OF REGENTS, Tucson, AZ (US)

(72) Inventors: Joe G.N. GARCIA, Tucson, AZ (US); Mohamed n. AHMED, Tucson, AZ (US)

(21) Appl. No.: 18/040,976

(22) PCT Filed: Aug. 6, 2021
(86) PCT No.: PCT/US2021/045005

§ 371 Date: Feb. 7, 2023

Related U.S. Application Data
(60) Provisional application No. 63/062,750, filed on Aug. 7, 2020.
(60) Provisional application No. 63/062,908, filed on Aug. 7, 2020.
(60) Provisional application No. 63/063,022, filed on Aug. 7, 2020.

(51) Int. Cl. C12Q 1/6883; C07K 16/40

(52) U.S. Cl. CPC C12Q 1/6883; C07K 16/40; C07K 2317/565

(57) ABSTRACT

A method of identifying single nucleotide polymorphisms (SNPs) within the NAMPT promoter that are associated with an inflammatory condition, such as cardiac ischemia, traumatic brain injury, cancer, chorioamnionitis, nonalcoholic steatohepatitis (NASH), or renal fibrosis. Also provided are methods of diagnosing and treating such inflammatory condition in a subject.

### RELATED APPLICATION

This application claims priority to U.S. Provisional Application No. 63/062,908, filed on Aug. 7, 2020, U.S. Provisional Application No. 63/063,022, filed on Aug. 7, 2020, and U.S. Provisional Application No. 63/062,750, filed on Aug. 7, 2020. The entire content of the foregoing priority applications is incorporated herein by reference.

### FEDERALLY-SPONSORED RESEARCH OR DEVELOPMENT

This invention was made with U.S. government support under grant number R41-HD101202-01 STTR by National Institutes of Health (NIH). The U.S. government has certain rights in the invention.

### SEQUENCE LISTING

The instant application contains a Sequence Listing which has been submitted electronically in ASCII format and is hereby incorporated by reference in its entirety. Said ASCII copy, created on Aug. 3, 2021, is named A110808_1010WO_SL_ST25 and is 16,363 bytes in size.

### FIELD OF THE INVENTION

This invention relates generally to the fields of inflammation and molecular biology. The invention provides methods of detecting single nucleotide polymorphisms (SNPs), methods for predicting the increased risk of developing inflammatory conditions, methods of diagnosing inflammatory condition, methods of treating the inflammatory condition, and methods of determining the responsiveness of a patient to a treatment, using SNPs.

### BACKGROUND

Inflammation is associated with a variety of conditions, such as cardiac ischemia, traumatic brain injury, cancer, chorioamnionitis, nonalcoholic steatohepatitis (NASH), and renal fibrosis. Cardiac ischemia, traumatic brain injury, and cancer are leading causes of morbidity and mortality worldwide. There is a need in the art for new and more effective methods of determining risk of, diagnosing, and treating such conditions.

Chorioamnionitis, also known as intra-amniotic infection (IAI), is a complication of pregnancy associated with possible maternal, perinatal, and long-term adverse outcomes. Chorioamnionitis is responsible for ˜40% of preterm labor cases (prematurity affects nearly 10% of pregnancies worldwide and is the most significant cause of perinatal mortality and morbidity). There is a need in the art for new and more effective methods of determining risk of, diagnosing, and treating chorioamnionitis.

Non-alcoholic fatty liver disease (NAFLD) affects up to 25% of the world's adult population and is associated with increased liver-related morbidity and mortality, increased risk of cardiovascular disease, extra-hepatic cancers, T2DM (1 in 8 dying of liver fibrosis/hepatocellular carcinoma) and chronic kidney disease. Despite growing prevalence, the factors influencing NAFLD development and subsequent progression from simple fatty liver (steatosis) to NASH, hepatic fibrosis/cirrhosis and hepatocellular carcinoma (HCC) (Anstee et al., Nat Rev Gastroenterol Hepatol (2019), 16:411-428) are poorly understood and novel therapeutic strategies for NAFLD treatment remains a continued unmet need. Increasing evidence indicates that mechanisms of NAFLD progression are complex and multifactorial involving genetic variations, lipid peroxidation, oxidative stress, imbalances in gut microbiota, with a very heavy implication for dysregulated innate immune responses (Diehl et al., N Engl J Med (2017), 377:2063-2072; Friedman et al., Nat Med (2018), 24:908-922). NASH affects an estimated 3% to 6% of the US population and the prevalence is increasing. An estimated 20% of patients with NASH will develop cirrhosis, and NASH is predicted to become the leading indication for liver transplants in the US. The mortality rate among patients with NASH is substantially higher than the general population or patients without this inflammatory subtype of NAFLD, with annual all-cause mortality rate of 25.56 per 1000 person-years and a liver-specific mortality rate of 11.77 per 1000 person-years. No NASH-specific therapies are approved by the US Food and Drug Administration. Thus, there is a need in the art for new and more effective methods of determining risk of, diagnosing, and treating NASH.

Renal fibrosis occurs when the normal renal tissue architecture is gradually replaced with extracellular matrix (ECM) (Meng et al., “Inflammatory processes in renal fibrosis,” Nat Rev Nephrol, 10:493-503 (2014)). Fibrosis of both glomerular and tubulointerstitial compartments correlates with declining renal function in all progressive forms of diabetic and nondiabetic glomerular disease, and is widely considered a common mechanism leading to end-stage renal disease (Bohle et al., “Serum creatinine concentration and renal interstitial volume. Analysis of correlations in endocapillary (acute) glomerulonephritis and in moderately severe mesangioproliferative glomerulonephritis,” Virchows Arch A Pathol Anat Histol, 375:87-96 (1977); Mackensen-Haen et al., “Correlations between renal cortical interstitial fibrosis, atrophy of the proximal tubules and impairment of the glomerular filtration rate,” Clin Nephrol 15:167-171 (1981); Seron et al., D., “Number of interstitial capillary cross-sections assessed by monoclonal antibodies: relation to interstitial damage,” Nephrol Dial Transplant, 5:889-893 (1990)). Renal inflammation is induced as a protective response to a wide range of injuries in an attempt to eliminate the cause and promote repair, but ongoing inflammation, regardless of the underlying etiology, promotes progressive renal fibrosis. However, the mechanisms underlying progressive renal fibrosis are not completely understood and, consequently, effective therapies are lacking. Thus, there is a need in the art for new and more effective methods of determining risk of, diagnosing, and treating renal fibrosis.

### SUMMARY OF THE INVENTION

Reducing the morbidity and mortality associated with inflammatory conditions includes identification of risk factors associated with disease progression; identification of markers indicative of disease; and/or development of novel approaches that attenuate disease severity and progression.

Regulation of innate immunity and reduction of inflammatory injury associated with an inflammatory condition, particularly, regulating NFkB-dependent inflammatory cascade, may be important for disease treatment and prevention.

In order to improve treatment and outcomes, biomarkers are needed that are indicative of inflammatory conditions and/or indicative of a risk of developing inflammatory conditions. Non-limiting examples of such markers include Single Nucleotide Polymorphisms (SNPs) in genes regulating cytokines such as nicotinamide phospho-ribosyltransferase enzyme (NAMPT), also called PBEF and visfatin.

Provided are methods of identifying single nucleotide polymorphisms (SNPs) within the NAMPT promoter that are associated with an inflammatory condition. At least some embodiments provide a means to identify patients who may be at risk for the inflammatory condition. Also provided are methods of diagnosing and treating the inflammatory condition.

Some embodiments comprise methods of identifying a subject having or at risk of developing cardiac ischemia, traumatic brain injury, cancer, chorioamnionitis, nonalcoholic steatohepatitis (NASH), or renal fibrosis, comprising, a) obtaining a sample from a subject at risk of having or developing cardiac ischemia, traumatic brain injury, cancer, chorioamnionitis, NASH, or renal fibrosis; and b) detecting the presence of at least one single nucleotide polymorphism (SNP) associated with the human nicotinamide phosphoribosyl transferase (NAMPT) gene in the sample, wherein the SNP is selected from the group consisting of rs7789066, rs61330082, rs9770242, rs59744560, rs116647506, rs61330082, rs114382471, and rs190893183. In some embodiments, the subject has cardiac ischemia, traumatic brain injury, cancer, chorioamnionitis, NASH, or renal fibrosis.

In some embodiments, the subject has at least 1 SNP, at least 2 SNPs, at least 3 SNPs, at least 4 SNPs, at least 5 SNPs, at least 6 SNPs, at least 7 SNPs, or at least 8 SNPs selected from the group consisting of rs7789066, rs61330082, rs9770242, rs59744560, rs116647506, rs61330082, rs114382471, and rs190893183. Some embodiments comprise detecting at least 2 SNPs selected from the group consisting of rs7789066, rs61330082, rs9770242, rs59744560, rs116647506, rs61330082, rs114382471, and rs190893183. Some embodiments comprise detecting at least one SNP selected from the group consisting of rs7789066, rs61330082, rs9770242, and rs59744560. Some embodiments comprise detecting at least one SNP selected from the group consisting of rs116647506, rs61330082, rs114382471, and rs190893183.

In some embodiments, the subject is of African descent.

In some embodiments, the detecting comprises using a polymerase chain reaction (PCR), a SNP microarray, SNP-restriction fragment length polymorphism (SNP-RFLP), dynamic allele-specific hybridization (DASH), primer extension (MALDI-TOF) mass spectrometry, single strand conformation polymorphism, and/or new generation sequencing (NGS).

In some embodiments, the detecting comprises contacting the sample with an oligonucleotide probe that selectively hybridizes to a nucleotide sequence comprising the SNP, or a nucleotide sequence complementary thereto, and detecting selective hybridization of the oligonucleotide probe. In certain embodiments, an oligonucleotide probe that selectively hybridizes to a nucleotide sequence comprising the SNP includes 200 base pairs on each side surrounding the SNP. In particular embodiments, an oligonucleotide probe comprising the nucleotide sequence set forth in SEQ ID NO: 18 selectively hybridizes to a nucleotide sequence comprising rs7789066; an oligonucleotide probe comprising the nucleotide sequence set forth in SEQ ID NO: 19 selectively hybridizes to a nucleotide sequence comprising rs61330082; an oligonucleotide probe comprising the nucleotide sequence set forth in SEQ ID NO: 20 selectively hybridizes to a nucleotide sequence comprising rs9770242; an oligonucleotide probe comprising the nucleotide sequence set forth in SEQ ID NO: 21 selectively hybridizes to a nucleotide sequence comprising rs59744560; and/or an oligonucleotide probe comprising the nucleotide sequence set forth in SEQ ID NO: 22 selectively hybridizes to a nucleotide sequence comprising rs1319501.

In some embodiments, the oligonucleotide probe comprises a detectable label, and wherein detecting selective hybridization of the probe comprises detecting the detectable label. In some embodiments, the detectable label comprises a fluorescent label, a luminescent label, a radionuclide, or a chemiluminescent label. In some embodiments, the oligonucleotide probe comprises a bi-labeled oligonucleotide probe, comprising a fluorescent moiety and a fluorescent quencher.

In some embodiments, the SNP is associated with a NAMPT promoter activity level that is higher than a baseline NAMPT promoter activity level. In certain embodiments, the baseline NAMPT promoter activity level is a level associated with a subject that does not have cardiac ischemia, traumatic brain injury, cancer, chorioamnionitis, NASH, or renal fibrosis.

In some embodiments, the sample is a plasma sample.

Also provided are methods of treating a subject having or at risk of having cardiac ischemia, traumatic brain injury, cancer, chorioamnionitis, NASH, or renal fibrosis by administering an effective amount of an NAMPT inhibitor to the subject having or at risk for developing cardiac ischemia, traumatic brain injury, cancer, chorioamnionitis, NASH, or renal fibrosis. In some embodiments, the subject may be determined to have or be at risk of having cardiac ischemia, traumatic brain injury, cancer, chorioamnionitis, NASH, or renal fibrosis based on one or more of a clinical diagnosis (e.g., identifying one or more symptoms and/or risk factors for the inflammatory condition), elevated levels of NAMPT (e.g., plasma NAMPT levels), and/or the identification of one or more SNPs associated with a NAMPT promoter activity level (e.g., rs7789066, rs61330082, rs9770242, rs59744560, rs116647506, rs61330082, rs114382471, and rs190893183).

Also provided are methods of treating a subject having or at risk of having cardiac ischemia, traumatic brain injury, cancer, chorioamnionitis, NASH, or renal fibrosis, said method comprising: a) obtaining a sample from a subject having or at risk of having cardiac ischemia, traumatic brain injury, cancer, chorioamnionitis, NASH, or renal fibrosis; b) detecting the presence or absence of at least one SNP in the sample, wherein the SNP is selected from the group consisting of rs7789066, rs61330082, rs9770242, rs59744560, rs116647506, rs61330082, rs114382471, and rs190893183, and wherein the presence of the at least one SNP indicates that the subject has or is at risk for developing cardiac ischemia, traumatic brain injury, cancer chorioamnionitis, NASH, or renal fibrosis; and c) administering an effective amount of an NAMPT inhibitor to the subject having or at risk for developing cardiac ischemia, traumatic brain injury, cancer, chorioamnionitis, NASH, or renal fibrosis. In some embodiments, the sample is a plasma sample.

Some embodiments comprise detecting at least 2 SNPs, at least 3 SNPs, at least 4 SNPs, at least 5 SNPs, at least 6 SNPs, at least 7 SNPS, or at least 8 SNPs selected from the group consisting of rs7789066, rs61330082, rs9770242, rs59744560, rs116647506, rs61330082, rs114382471, and rs190893183. In some embodiments, detecting the presence or absence of at the least one SNP comprises detecting the presence or absence of at least one SNP selected from the group consisting of rs7789066, rs61330082, rs9770242 and rs59744560. In some embodiments, detecting the presence or absence of at the least one SNP comprises detecting the presence or absence of at least one SNP selected from the group consisting of rs116647506, rs61330082, rs114382471, and rs190893183.

In some embodiments, the treated subject is of African descent.

In some embodiments, the detecting comprises using polymerase chain reaction (PCR), a SNP microarray, SNP-restriction fragment length polymorphism (SNP-RFLP), dynamic allele-specific hybridization (DASH), primer extension (MALDI-TOF) mass spectrometry, single strand conformation polymorphism, or new generation sequencing (NGS). In some embodiments, the detecting comprises contacting the sample with an oligonucleotide probe that selectively hybridizes to a nucleotide sequence comprising the SNP, or a nucleotide sequence complementary thereto, and detecting selective hybridization of the oligonucleotide probe. In certain embodiments, an oligonucleotide probe that selectively hybridizes to a nucleotide sequence comprising the SNP includes 200 base pairs on each side surrounding the SNP. In particular embodiments, an oligonucleotide probe comprising the nucleotide sequence set forth in SEQ ID NO: 18 selectively hybridizes to a nucleotide sequence comprising rs7789066; an oligonucleotide probe comprising the nucleotide sequence set forth in SEQ ID NO: 19 selectively hybridizes to a nucleotide sequence comprising rs61330082; an oligonucleotide probe comprising the nucleotide sequence set forth in SEQ ID NO: 20 selectively hybridizes to a nucleotide sequence comprising rs9770242; an oligonucleotide probe comprising the nucleotide sequence set forth in SEQ ID NO: 21 selectively hybridizes to a nucleotide sequence comprising rs59744560; and/or an oligonucleotide probe comprising the nucleotide sequence set forth in SEQ ID NO: 22 selectively hybridizes to a nucleotide sequence comprising rs1319501.

In some embodiments, the oligonucleotide probe comprises a detectable label, and wherein detecting selective hybridization of the probe comprises detecting the detectable label. In some embodiments, the detectable label comprises a fluorescent label, a luminescent label, a radionuclide, or a chemiluminescent label. In some embodiments, the oligonucleotide probe comprises a bilabeled oligonucleotide probe, comprising a fluorescent moiety and a fluorescent quencher.

In some embodiments, the SNP is associated with a NAMPT promoter activity level that is higher than a baseline NAMPT promoter activity level. In some embodiments, the baseline NAMPT promoter activity level is a level associated with a subject that does not have cardiac ischemia, traumatic brain injury, cancer, chorioamnionitis, NASH, or renal fibrosis.

In some embodiments, the NAMPT inhibitor is an anti-NAMPT antibody.

In some embodiments, the anti-NAMPT antibody comprises: CDR1, CDR2, and CDR3 domains of a heavy chain variable region having an amino acid sequence set forth as SEQ ID NO: 2; and CDR1, CDR2, and CDR3 domains of a light chain variable region having an amino acid sequence set forth as SEQ ID NO: 3. In certain embodiments, the CDR1, CDR2, and CDR3 domains of the heavy chain variable region have amino acid sequences set forth as SEQ ID NOs: 4, 5, and 6, respectively; and/or the CDR1, CDR2, and CDR3 domains of the light chain variable region have amino acid sequences set forth as SEQ ID NOs: 7, 8, and 9, respectively.

In some embodiments, the heavy chain variable region comprises the CDR1, CDR2, and CDR3 domains of the amino acid sequence set forth in SEQ ID NO: 2. In some embodiments, the light chain variable region comprises the CDR1, CDR2, and CDR3 domains of the amino acid sequence set forth in SEQ ID NO: 3. In some embodiments, the heavy chain variable region comprises the amino acid sequence set forth in SEQ ID NO: 2, and/or the light chain variable region comprises the amino acid sequence set forth in SEQ ID NO: 3.

In some embodiments, the anti-NAMPT antibody comprises: CDR1, CDR2, and CDR3 domains of a heavy chain variable region having an amino acid sequence set forth as SEQ ID NO: 10; and CDR1, CDR2, and a CDR3 domains of a light chain variable region having an amino acid sequence set forth as SEQ ID NO: 11. In certain embodiments, the CDR1, CDR2, and CDR3 domains of the heavy chain variable region have amino acid sequences set forth as SEQ ID NOs: 12, 13, and 14, respectively; and/or the CDR1, CDR2, and CDR3 domains of the light chain variable region have amino acid sequences set forth as SEQ ID NOs: 15, 16, and 17, respectively.

In some embodiments, the heavy chain variable region comprises the CDR1, CDR2, and CDR3 domains of the amino acid sequence set forth in SEQ ID NO: 10. In some embodiments, the light chain variable region comprises the CDR1, CDR2, and CDR3 domains of the amino acid sequence set forth in SEQ ID NO: 11. In some embodiments, the heavy chain variable region comprises the amino acid sequence set forth in SEQ ID NO: 10, and/or the light chain variable region comprises the amino acid sequence set forth in SEQ ID NO: 11.

In some embodiments, the anti-NAMPT antibody comprises: CDR1, CDR2, and CDR3 domains of a heavy chain variable region having an amino acid sequence set forth as SEQ ID NO: 23; and CDR1, CDR2, and CDR3 domains of a light chain variable region having an amino acid sequence set forth as SEQ ID NO: 3. In certain embodiments, the CDR1, CDR2, and CDR3 domains of the heavy chain variable region have amino acid sequences set forth as SEQ ID NOs: 4, 24, and 6, respectively; and/or the CDR1, CDR2, and CDR3 domains of the light chain variable region have amino acid sequences set forth as SEQ ID NOs: 7, 8, and 9, respectively.

In some embodiments, the heavy chain variable region comprises the CDR1, CDR2, and CDR3 domains of the amino acid sequence set forth in SEQ ID NO: 23. In some embodiments, the light chain variable region comprises the CDR1, CDR2, and CDR3 domains of the amino acid sequence set forth in SEQ ID NO: 3. In some embodiments, the heavy chain variable region comprises the amino acid sequence set forth in SEQ ID NO: 23, and/or the light chain variable region comprises the amino acid sequence set forth in SEQ ID NO: 3.

In some embodiments, the subject has or is at risk of developing cardiac ischemia. In some embodiments, the subject has or is at risk of developing traumatic brain injury. In other embodiments, the subject has or is at risk of developing cancer. In yet other embodiments, the subject has or is at risk of developing chorioamnionitis. In some embodiments, the subject has or is at risk of developing NASH. In some embodiments, the subject has or is at risk of developing renal fibrosis.

### BRIEF DESCRIPTION OF THE DRAWINGS

FIG. 1 is a schematic showing secretion of NAMPT in response to a variety of injurious signals, binding of extracellular NAMPT to TLR4 for activation of innate immunity and inflammation, and blocking of inflammation by neutralizing NAMPT with the anti-NAMPT mAb, ALT-100.

FIG. 2 provides representative micrographs showing immunohistochemical (IHC) staining for NAMPT in placental tissues obtained from healthy control women (FIG. 2, two left panels) or women with chorioamnionitis (FIG. 2, two right panels).

FIG. 3 is a graphical representation of NAMPT levels in plasma samples obtained from healthy pregnant women (“Pregnant”), pregnant women with chorioamnionitis (“ChorP”) or non-pregnant women controls (“Control”).

FIGS. 4A-4B depict chorioamnionitis-related uterine histology in a murine model of LPS-induced chorioamnionitis. FIG. 4A provides representative micrographs showing uterine abscess (FIG. 4A, top panel), and areas of inflammation and apoptosis (FIG. 4A, bottom panel) in uterine tissues obtained from LPS-challenged chorioamnionitis mice. FIG. 4B provides graphs showing levels of pro-inflammatory cytokines IL-1B, IL-6, KC (IL-8) and MCP1 in serum (FIG. 4B, top panel) or uterine tissue homogenates (FIG. 4B, bottom panel) derived from LPS-challenged chorioamnionitis mice.

FIGS. 5A-5C depict effects of a humanized anti-NAMPT monoclonal antibody (mAb) ALT-100 on expression of NAMPT and other pro-inflammatory cytokines in pups born to chorioamnionitic mice, and on percent survival of pups born to chorioamnionitic mice. FIG. 5A provides representative images from western blot analyses showing NAMPT expression in lung tissue of pups born to anti-NAMPT mAb (ALT-100)-treated chorioamnionitic mice, PBS-treated chorioamnionitic mice or healthy mouse controls. FIG. 5B is a graphical representation of levels of pro-inflammatory cytokines IL-6, KC (IL-8) and MCP1 in lung tissue homogenate of pups born to anti-NAMPT mAb-treated chorioamnionitic mice (“LPS+ALT-100”) or PBS-injected chorioamnionitic mice (“LPS”). FIG. 5C is a graphical representation of percent survival of pups born to PBS-challenged control mice (“Control”), LPS-challenged chorioamnionitic mice (“LPS”), or LPS-challenged chorioamnionitic mice treated with anti-NAMPT mAb (“LPS+ALT-100”). * indicates p<0.05.

FIGS. 6A-6C depict effects of a humanized anti-NAMPT mAb ALT-100 on inflammation and bronchopulmonary dysplasia (BPD), as evaluated by H&E staining, in lung tissues obtained from a murine model of BPD. FIG. 6A is a representative micrograph showing H&E staining in lung tissue obtained from pups exposed to control room air. FIG. 6B is a representative micrograph showing H&E staining in lung tissue obtained from LPS/hyperoxia-challenged BPD pups. FIG. 6C is a representative micrograph showing H&E staining in lung tissue obtained from LPS/hyperoxia-challenged BPD pups that were treated with anti-NAMPT mAb.

FIGS. 7A-7B depict NAMPT expression, as evaluated by western blot analysis, in neonatal lung tissues obtained from mice exposed to LPS and to hyperoxia in a murine model of BPD. FIG. 7A provides representative images showing western blot analysis of NAMPT expression in lung tissue of pups that were exposed to control room air (“Control”), to LPS/hyperoxia (“LPS/Hyperoxia”), or to LPS/hyperoxia but treated with anti-NAMPT Ab (“LPS/Hyperoxia+anti-NAMPT Ab”). FIG. 7B is a graphical representation of densitometric measurements of NAMPT band intensity normalized for GAPDH in lung tissue of pups that were exposed to control room air (“Control”), LPS/hyperoxia challenged pups (“LPS/Hyperoxia”), or LPS/hyperoxia challenged pups that were treated with anti-NAMPT mAb (“LPS/Hyperoxia+ALT-100”).

FIGS. 8A-8B depict effects of a humanized anti-NAMPT mAb (ALT-100) on chorioamnionitis-induced pulmonary hypertension (PAH), as evaluated by RT-PCR analysis, in lung tissue homogenates obtained from a two-hit murine model of chorioamnionitis-induced PAH. FIG. 8A is a graphical representation of p-SMAD 1/5/8/9 expression in lung tissues from pups that were exposed to control room air (“RA”), pups that were challenged with LPS at room air (“RA LPS”), LPS challenged pups that were treated with anti-NAMPT mAb (“RA LPS NAMPT”), pups that were challenged with LPS and hyperoxia (“HO LPS”), or LPS/hyperoxia challenged pups that were treated with anti-NAMPT mAb (“HO LPS NAMPT”). FIG. 8B is a graphical representation of STATS/GAPDH fold change in lung tissues from pups that were exposed to control room air (“RA”), pups that were challenged with LPS at room air (“LPS RA”), LPS challenged pups that were treated with anti-NAMPT mAb (“LPS NAMPT RA”), pups that were challenged with LPS and hyperoxia (“LPS Hyperoxia”), or LPS/hyperoxia challenged pups that were treated with anti-NAMPT mAb (“LPS NAMPT Hyperoxia”).

FIG. 9 is a graphical representation of Fulton index in pups from a two-hit murine model of chorioamnionitis-induced PAH, and depicts RV/LV septum ratio in pups that were exposed to control room air, pups that were challenged with LPS and hyperoxia, or LPS/hyperoxia challenged pups that were treated with anti-NAMPT mAb.

FIGS. 10A-10E provide representative images showing NAMPT expression in hepatic tissues from human non-alcoholic fatty liver disease (NAFLD), murine nonalcoholic steatohepatitis (NASH) tissues, and murine fatty liver tissues. FIG. 10A provides representative micrographs showing IHC staining for NAMPT in liver sections from NAFLD subjects (n=4-5) as compared to normal liver tissues (inset). FIG. 10B provides representative micrographs showing IHC staining for NAMPT in liver sections from murine STAM model of NASH (STZ/HF) taken at 6 weeks (n=8), as compared to normal liver tissues (inset). FIG. 10C provides representative micrographs showing IHC staining for NAMPT in liver sections of mice from a western diet-induced NASH model that were fed high fat high sucrose (HFHS) (8 weeks, sacrificed at 16 weeks), as compared to normal liver tissues (inset). FIG. 10D provides representative images showing western blot analysis of NAMPT expression in liver tissue lysates from normal diet and HFHS fed mice (16 weeks, n=4 mice). FIG. 10E provides representative micrographs showing IHC staining for NAMPT in normal liver sections and liver sections from mouse with fatty liver disease.

FIGS. 11A-11B depict the effects of a humanized anti-NAMPT mAb on NASH-mediated hepatic injury, as assessed by hematoxylin and eosin (H&E) staining, in liver tissues obtained from streptozotocin-challenged NASH mice that were maintained on a high fat diet (STZ/HF (STAM) mice). FIG. 11A provides representative micrographs showing evidence of steatosis in vehicle (IgG₁ Ab)-treated STZ/HF (STAM) mice (left panel), as compared to control mice (left panel, inset) at 12 weeks, and reduction in steatosis, intrahepatic fat globules, hepatocyte injury and ballooning in anti-NAMPT mAb treated STZ/HF mice (right panel). FIG. 11B provides magnified version (200×) of the images from FIG. 10A.

FIGS. 12A-12D depict effects of a humanized anti-NAMPT mAb on multiple indices of STZ/HF-induced murine NASH (STAM). FIG. 12A is a graphical representation of steatosis score, as assessed in liver tissues from anti-NAMPT mAb treated STZ/HF mice (“mAb”; n=8), vehicle injected STZ/HF mice (“Veh”; n=8) or Telmisartan-treated STZ/HF mice (“Telm”; n=8). FIG. 12B is a graphical representation of liver triglyceride level, as assessed in liver tissues from anti-NAMPT mAb treated STZ/HF mice (“mAb”; n=8), vehicle injected STZ/HF mice (“Veh”; n=8) or Telmisartan-treated STZ/HF mice (“Telm”; n=8). FIG. 12C is a graphical representation of Sirius staining for collagen, a marker of hepatic fibrosis, as assessed in liver tissues from anti-NAMPT mAb treated STZ/HF mice (“mAb”; n=8), vehicle injected STZ/HF mice (“Veh”; n=8) or Telmisartan-treated STZ/HF mice (“Telm”; n=8). FIG. 12D is a graphical representation of NAMPT plasma levels, as assessed in anti-NAMPT mAb treated STZ/HF mice (“mAb”), vehicle injected STZ/HF mice (“Veh”), Telmisartan-treated STZ/HF mice (“Telm”), or control mice (“Ctrl”) that were not exposed to STZ/HF. * p<0.01, #p<0.0001.

FIG. 13 is a schematic showing NAMPT as a novel NASH therapeutic target. The left panel shows the role of NAMPT in regulating inflammation and fibrosis in NASH. The right panel shows protective effects of neutralizing anti-NAMPT mAb, ALT-100.

FIGS. 14A-14C depict the effects of a humanized anti-NAMPT mAb ALT-100 on the severity of renal fibrosis in a murine model of unilateral ureteral obstruction (UUO). FIG. 14A provides representative micrographs showing Sirius red-staining/collagen deposition in renal tissues of UUO mice at day 14 post UUO, as compared to control tissues (inset). FIG. 14B provides representative micrograph showing Sirius red-staining/collagen deposition in renal tissues from UUO mice that were treated with anti-NAMPT mAb ALT-100. FIG. 14C is a graphical representation of Sirius red-staining/collagen deposition in normal renal tissue, renal tissue from UUO mice, and renal tissue from UUO mice that were treated with anti-NAMPT mAb ALT-100. * p<0.0001.

### DETAILED DESCRIPTION OF THE INVENTION

### Definitions

In order that the invention may be more readily understood, certain terms are first defined. In addition, it should be noted that whenever a value or range of values of a parameter are recited, it is intended that values and ranges intermediate to the recited values are also part of this invention. It is also to be noted that as used herein, the singular forms “a,” “and” and “the” include plural references unless the context clearly dictates otherwise.

The term “single nucleotide polymorphism,” or “SNP,” as used interchangeably here, refers to a DNA sequence variation occurring when a single nucleotide in the genome (or other shared sequence) differs between members of a species (or between paired chromosomes in an individual). A SNP can occur in either a coding or non-coding region of the genome of an organism.

The term “NAMPT” or “eNAMPT”, used interchangeably herein, refers to the secreted form of nicotinamide phosphoribosyltransferase (NAMPT) unless specifically mentioned to relate to a non-secreted form (e.g., intracellular NAMPT or NAMPT nucleic acids). The amino acid sequence of secreted human NAMPT (also referred to as human eNAMPT) is provided below as SEQ ID NO: 1 (see also NCBI Gene Ref. No. NC_000007.14 and Protein Ref. No. NP_005737.1).

(SEQ ID NO: 1)

MNPAAEAEFN ILLATDSYKV THYKQYPPNT SKVYSYFECR
EKKTENSKLR KVKYEETVFY GLQYILNKYL KGKVVTKEKI
QEAKDVYKEH FQDDVFNEKG WNYILEKYDG HLPIEIKAVP
EGFVIPRGNV LFTVENTDPE CYWLTNWIET ILVQSWYPIT
VATNSREQKK ILAKYLLETS GNLDGLEYKL HDFGYRGVSS
QETAGIGASA HLVNFKGTDT VAGLALIKKY YGTKDPVPGY
SVPAAEHSTI TAWGKDHEKD AFEHIVTQFS SVPVSVVSDS
YDIYNACEKI WGEDLRHLIV SRSTQAPLII RPDSGNPLDT
VLKVLEILGK KFPVTENSKG YKLLPPYLRV IQGDGVDINT
LQEIVEGMKQ KMWSIENIAF GSGGGLLQKL TRDLLNCSFK
CSYVVTNGLG INVFKDPVAD PNKRSKKGRL SLHRTPAGNF
VTLEEGKGDL EEYGQDLLHT VFKNGKVTKS YSFDEIRKNA
QLNIELEAAHH

NAMPT is also referred to as pre-B cell colony enhancing factor (PBEF) or visfatin.

The term “baseline”, as used herein, refers to a reference or control measurement, e.g., a control level of NAMPT expression from a healthy subject (i.e., a subject not having the inflammatory condition) or group of healthy subjects.

As used herein, a “NAMPT inhibitor” or an “inhibitor of NAMPT” refers to an agent that reduces or prevents NAMPT activity. In some embodiments, a NAMPT inhibitor binds to NAMPT, resulting in inhibition of the biological activity of NAMPT.

As used herein, the terms “NAMPT antibody” or “anti-NAMPT antibody” or “anti-eNAMPT antibody,” used interchangeably herein, refer to an antibody that specifically binds to the secreted form of NAMPT (also referred to herein as eNAMPT). In a preferred embodiment, the antibody specifically binds to human NAMPT (hNAMPT). Preferably, NAMPT antibodies inhibit the biological activity of NAMPT. It will be appreciated that modified NAMPT activity may be measured directly using art recognized techniques or may be measured by the impact the altered activity has downstream.

The term “level” or “amount” as used herein refers to the measurable quantity of a biomarker, e.g., a level of NAMPT expression. The amount may be either (a) an absolute amount as measured in molecules, moles, or weight per unit volume or cells or (b) a relative amount, e.g., measured by densitometric analysis.

The term “sample” as used herein refers to material (e.g., a collection of similar cells or tissue) obtained from a subject. The sample may be solid tissue (e.g., placental tissue) as from a fresh, frozen and/or preserved organ or tissue sample or biopsy or aspirate; blood or any blood constituents; or bodily fluids, such as blood, serum, plasma, urine, saliva, sweat or synovial fluid. In some embodiments, the synovitis biomarker is obtained from a serum sample. In some embodiments, the cartilage degradation biomarker is obtained from a urine sample.

The terms “patient,” “individual,” or “subject” are used interchangeably herein, and refer to a mammal, particularly, a human (e.g., a pregnant female human). The patient may have no disease, mild, intermediate or severe disease. The patient may be treatment naïve, responding to any form of treatment, or refractory. The patient may be an individual in need of treatment or in need of diagnosis based on particular symptoms or family history. In some embodiments, a subject is a human subject that has been diagnosed with, or has symptoms of, an inflammatory condition. In other embodiments, a subject is a healthy human subject that has not been diagnosed with, or does not have symptoms of, the inflammatory condition.

Terms such as “treating” or “treatment” or “to treat” or “alleviating” or “to alleviate” refer to therapeutic measures that cure, slow down, lessen symptoms of, and/or halt or slow the progression of a condition or disorder.

Terms such as “prevent,” and the like refer to prophylactic or preventative measures that prevent the development of a targeted condition.

The term “effective amount” or “therapeutically effective amount” are used interchangeably herein, and refer to an amount of an agent that is effective to achieve a particular biological result. Such results may include, but are not limited to, the inhibition of NAMPT expression or activity, or the expression or activity of signaling molecules which are downstream of NAMPT as determined by any means suitable in the art.

### Diseases/Conditions

Provided are methods of determining risk of developing conditions associated with NAMPT expression. Also provided are methods of diagnosing and/or treating such conditions. In some embodiments, the NAMPT-associated condition is an inflammatory condition, such as cardiac ischemia, myocardial infarction, traumatic brain injury, cancer, chorioamnionitis, nonalcoholic steatohepatitis (NASH), or renal fibrosis. In some embodiments, the condition is a condition described in WO/2021/026487 to Garcia, published on Feb. 11, 2021, which is herein incorporated by reference in its entirety (e.g., acute respiratory distress syndrome (ARDS), radiation-induced lung injury (RILI), pulmonary hypertension, pulmonary fibrosis, COVID-19, or prostate cancer).

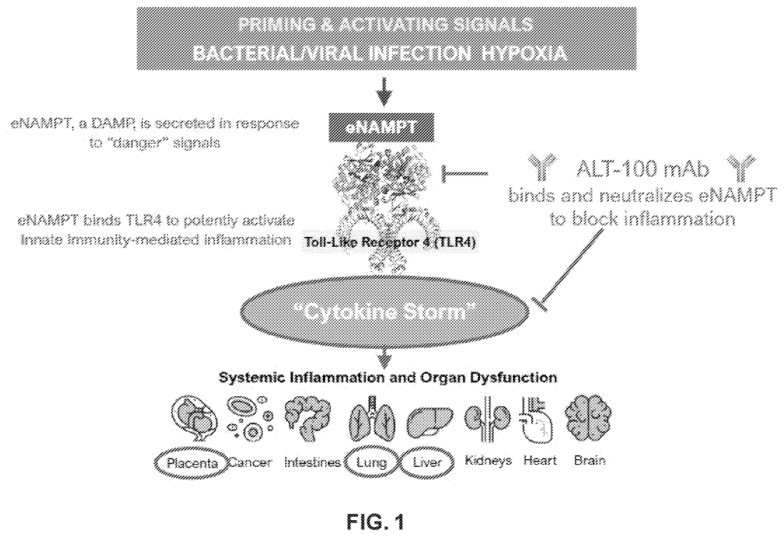

Caloric excess and sedentary lifestyles have led to a global epidemic of obesity, metabolic syndrome and nonalcoholic fatty liver disease (NAFLD), which has emerged as a leading cause of chronic liver disease (Ali and Cusi, “New diagnostic and treatment approaches in non-alcoholic fatty liver disease (NAFLD),” Ann Med, 41:265-278 (2009); Younossi et al., “Global burden of NAFLD and NASH: trends, predictions, risk factors and prevention,” Nat Rev Gastroenterol Hepatol, 15:11-20 (2018)). NAFLD represents a spectrum that progressively ranges from simple fatty liver (steatosis) to NASH, hepatic fibrosis/cirrhosis and an increased risk of hepatocellular carcinoma (HCC) (Adams et al., “The natural history of nonalcoholic fatty liver disease: a population-based cohort study,” Gastroenterology, 129:113-121 (2005); Anstee et al., “From NASH to HCC: current concepts and future challenges,” Nat Rev Gastroenterol Hepatol, 16:411-428 (2019); White et al., “Association between nonalcoholic fatty liver disease and risk for hepatocellular cancer, based on systematic review,” Clin Gastroenterol Hepatol, 10:1342-1359 e1342 (2012)). Despite growing prevalence, the factors influencing NAFLD development and subsequent progression are not well understood. Therapeutic advances in the complex pathogenesis of progressive NAFLD now highlight the role of innate immunity-driven inflammatory responses and oxidative/proteotoxic stress (Febbraio et al., “Preclinical Models for Studying NASH-Driven HCC: How Useful Are They?” Cell Metab, 29:18-26 (2019)) in NAFLD progression to NASH. Utilizing systems biology and genomic strategies, the present inventors have identified NAMPT as a novel damage-associated molecular pattern protein (DAMP) that potently regulates innate immunity-mediated inflammation and fibrosis via activation of TLR4-mediated inflammatory pathways (Ye et al., “Pre-B-cell colony-enhancing factor as a potential novel biomarker in acute lung injury,” Am J Respir Crit Care Med, 171:361-370 (2005); Camp et al., “Unique Toll-Like Receptor 4 Activation by NAMPT/PBEF Induces NFkappaB Signaling and Inflammatory Lung Injury,” Sci Rep, 5:13135 (2015); Oita et al., “Novel Mechanism for Nicotinamide Phosphoribosyltransferase Inhibition of TNF-alpha-mediated Apoptosis in Human Lung Endothelial Cells,” Am J Respir Cell Mol Biol, 59:36-44 (2018)). Circulating NAMPT is an essential participant in the development and severity of multi-organ inflammation, injury and fibrosis (lung, liver, renal, cardiac) and as well as specific cancers (Sun et al., “Role of secreted extracellular nicotinamide phosphoribosyltransferase (eNAMPT) in prostate cancer progression: Novel biomarker and therapeutic target,” EBioMedicine, 61:103059 (2020)). Preliminary preclinical studies with NAMPT-neutralizing humanized ALT-100 mAb indicate that neutralization of circulating NAMPT reduces TLR4 inflammatory cascade activity and the severity of acute respiratory distress syndrome (ARDS), ventilator-induced lung injury (VILI), trauma-induced lung injury, pulmonary hypertension, radiation pneumonitis, lung fibrosis, renal fibrosis, cardiac fibrosis, and prostate cancer (Sun et al., “Role of secreted extracellular nicotinamide phosphoribosyltransferase (eNAMPT) in prostate cancer progression: Novel biomarker and therapeutic target,” EBioMedicine, 61:103059 (2020); Hong et al., “Essential role of pre-B-cell colony enhancing factor in ventilator-induced lung injury,” Am J Respir Crit Care Med, 178:605-617 (2008); Quijada et al., “Endothelial eNAMPT Amplifies Preclinical Acute Lung Injury: Efficacy of an eNAMPT-Neutralising mAb,” Eur Respir J, 2002536 (2020); Chen et al., “Nicotinamide Phosphoribosyltransferase Promotes Pulmonary Vascular Remodeling and Is a Therapeutic Target in Pulmonary Arterial Hypertension,” Circulation, 135:1532-1546 (2017); Sun et al., “Direct Extracellular NAMPT Involvement in Pulmonary Hypertension and Vascular Remodeling. Transcriptional Regulation by SOX and HIF-2alpha,” Am J Respir Cell Mol Biol, 63:92-103 (2020)). This is depicted in FIG. 1. As described in FIG. 1, NAMPT is secreted in response to a variety of injurious signals (e.g., infection (bacterial, viral, etc.), hypoxia, lipotoxicity, ROS, etc.) to potentially produce organ-confined inflammation (e.g., in placenta, lung, liver, prostate, kidneys, heart) or multi-organ inflammation and injury as a consequence of “cytokine storm” as in acute inflammatory lung disease, such as ARDS. NAMPT functions as a DAMP by binding to TLR4 and triggering the TLR4 inflammatory cascade, which contribute to NAFLD transition to NASH. Thus, the humanized NAMPT-neutralizing mAb, ALT-100, may dampen inflammatory cascades and ameliorate, delay and/or inhibit various inflammatory indications, such as acute indications (e.g., ARDS, trauma-induced brain and/or lung injury, intra-uterine infection and/or premature birth (e.g., those associated with chorioamnionitis), cardiac ischemia, etc.), acute and chronic (e.g., RILI), and chronic indication indications (e.g., pulmonary hypertension, pulmonary fibrosis, NASH, hepatic fibrosis, renal fibrosis, prostate cancer, etc.). For example, humanized NAMPT-neutralizing mAb, ALT-100, may delay or inhibit transition from NAFLD to an aggressive NASH phenotype with associated lethality.

Cardiac ischemia (also called myocardial ischemia) is a condition characterized by reduced blood flow to the heart. Cardiac ischemia reduces the heart muscle's ability to pump blood and is believed to result from a partial or complete blockage of the heart's arteries. Symptoms of cardiac ischemia include one or more of chest pressure or pain, typically on the left side of the body (angina pectoris); neck and/or jaw pain; should and/or arm pain; a fast heartbeat; shortness of breath, especially accompanying physical activity; nausea; vomiting; sweating; and fatigue. Without being bound by theory, it is believed that cardiac ischemia can be caused by coronary artery disease (atherosclerosis), blood clots, or coronary artery spasms; and it can be triggered by physical exertion, emotional stress, cold temperatures, cocaine use, eating a heavy or large meal, and/or sexual intercourse. Risk factors for cardiac ischemia include tobacco use, diabetes, high blood pressure, high blood cholesterol levels, high blood triglyceride levels, obesity, large waist circumference, and/or lack of physical activity. Adverse outcomes associated with cardiac ischemia include heart attack, irregular heart rhythm (arrhythmia), and/or heart failure. Cardiac ischemia is discussed in greater detail in Shimokawa et al., “Myocardial ischemia: Current concepts and future perspectives,” J. Cardiology, 52(2): 67-78 (2008), which is incorporated herein by reference in its entirety.

Myocardial infarction, also known as a heart attack, occurs when flow of blood to the heart is blocked. Without being bound by theory, it is believed that the blockage is most often a buildup of fat, cholesterol, and other substances, which form a plaque in the coronary arteries. Symptoms of myocardial infarction include one or more of pressure, tightness, pain, or a squeezing or aching sensation in the chest or arms that may spread to the neck, jaw, or back; nausea; indigestion; heartburn; abdominal pain; shortness of breath; cold sweat; fatigue; lightheadedness; and sudden dizziness. Risk factors for myocardial infarction include age (e.g., men age 45 and older; women age 55 and older); tobacco use; high blood pressure; high blood cholesterol or triglyceride levels; obesity; diabetes; metabolic syndrome; family history of heart attacks; lack of physical activity; stress; illicit drug use; history of preeclampsia; and autoimmune conditions. Adverse outcomes associated with myocardial infarction include arrhythmia; heart failure; and cardiac arrest. Myocardial infarction is discussed in greater detail in Saleh et al, “Understanding myocardial infarction,” F1000 Research, 7(F1000 Faculty Rev): 1378 (8 pages) (2018).

Traumatic brain injury is an injury to the brain caused by a blow or jolt to the head or body, or by an object that penetrates brain tissue. Traumatic brain injury can result in bruising, torn tissues, bleeding, and other physical damage to the brain. Without being bound by theory, it is believed that traumatic brain injury causes include falls, vehicle-related collisions, violence, sports injuries, and explosive blasts and other combat injuries. Subjects most at risk of traumatic brain injury include children (especially newborns to 4-year olds), young adults (especially between the ages of 15-24); adults age 60 and older; and males. Symptoms of traumatic brain injury include one or more of loss of consciousness from several minutes to hours; persistent headache or headache that worsens; repeated vomiting or nausea; convulsions or seizures; dilation of one or both pupils of the eyes; clear fluids draining from the nose or ears; inability to awaken from sleep; weakness or numbness in fingers and toes; loss of coordination; profound confusion; agitation, combativeness or other unusual behavior; slurred speech; coma and other disorders of consciousness; change in eating or nursing habits; unusual or easy irritability; persistent crying and inability to be consoled; change in ability to pay attention; change in sleep habits; sad or depressed mood; drowsiness; and loss of interest in favorite toys or activities. Traumatic brain injury is discussed in more detail in Ng et al., “Traumatic Brain Injuries: Pathophysiology and Potential Therapeutic Targets,” Front. Cell. Neurosci., 13 (Article 528): 1-23 (2019), which is incorporated herein by reference in its entirety.

Inflammation is often associated with the development and progression of cancer. Without being bound by theory, it is believed that cancer-associated inflammation can be caused by tumor-extrinsic factors, such as bacterial and viral infections, autoimmune diseases, obesity, tobacco smoking, asbestos exposure, and/or excessive alcohol consumption; or by tumor-intrinsic factors, such as cancer-initiated mutations. Cancer-associated inflammation is discussed in more detail in Singh et al., “Inflammation and Cancer,” Annals of African Medicine, 18(3): 121-126 (2019) and Greten et al., “Inflammation and Cancer: Triggers, Mechanisms, and Consequences,” Immunity Review, 51: 27-41 (2019), both of which are incorporated herein by reference in their entireties.

Chorioamnionitis is a condition that occurs before or during labor, or after delivery, and it can be characterized by neutrophilic infiltration and/or inflammation at the maternal fetal interface. It can be acute, subacute, or chronic. Chorioamnionitis is traditionally associated with a bacterial infection, for instance in the setting of membrane rupture, and can be associated with acute inflammation of the membranes and/or chorion of the placenta. Chorioamnionitis symptoms include fever, sweating, vaginal discharge, discolored and foul-smelling amniotic fluid, low glucose levels in amniotic fluid, high concentration of white blood cells and/or bacteria in amniotic fluid, uterine tenderness, maternal leukocytosis, purulent cervical drainage, and/or fetal tachycardia.

Chorioamnionitis can result in a variety of adverse consequences for either or both of a pregnant subject (e.g., a subject delivering a newborn) or the newborn subject. For instance, chorioamnionitis can result in chronic lung disease, brain injury, cerebral palsy, retinopathy of prematurity, respiratory distress syndrome, bronchopulmonary dysplasia, very low birth weight (i.e., less than 1,500 grams), neonatal sepsis, meningitis, pneumonia, impaired brain development, stillbirth, and/or death in a fetus or infant whose birth mother had chorioamnionitis. Adverse maternal outcomes include postpartum infections, sepsis, bacteremia, endometritis, need for cesarean delivery, heavy blood loss with delivery, blood clots in lungs and/or pelvis, and/or death.

Chorioamnionitis is discussed in greater detail in Cappelletti et al., “Immunobiology of Acute Chorioamnionitis,” Front. Immunol., 11(649): 1-21 (2020); Tita et al., “Diagnosis and Management of Clinical Chorioamnionitis,” Clin. Perinatol., 37(2): 339-54 (2010); and Committee on Obstetric Practice, “Intrapartum Management of Intraamniotic Infection,” The American College of Obstetricians and Gynecologists: AGOG Committee Opinion No. 712: 7 page (2017), each of which are incorporated herein by reference in their entireties.

NASH is a condition characterized by liver inflammation and damage caused by a buildup of fat in the liver. It is characterized by fatty change with lobular inflammation, hepatocellular injury, and Mallory hyalin, with or without fibrosis, in the absence of excessive alcohol consumption. NASH symptoms include enlarged liver, darkened skin patches (e.g., over knuckles, elbows, and/or knees), fatigue, itching, pain in upper right abdomen, bruising and bleeding easily, spider-like blood vessels just beneath the skin's surface, swollen belly, enlarged blood vessels underneath the skin, enlarged breasts in men, red palms, hepatic encephalopathy, and/or jaundice (i.e., yellowish skin and eyes).

Without being bound by theory, NASH is believed to be caused by one or more of being overweight or obese, having insulin resistance, having abnormal levels of fats in the blood (e.g., high levels of triglycerides, high levels of total cholesterol, high levels of LDL cholesterol, low levels of HDL cholesterol), having dyslipidemia, having hypertriglyceridemia, having a metabolic syndrome or one or more traits of a metabolic syndrome, having type 2 diabetes, having high blood pressure, having a disorder that causes the body to use or store fat improperly, rapid weight loss, certain infections (such as hepatitis C), certain medicines (such as amiodarone, diltiazem, glucocorticoids, highly active antiretroviral therapy, methotrexate, synthetic estrogens, tamoxifen, or valproic acid), and exposure to toxins. Each of the above can be considered risk factors for NASH.

NASH can result in a variety of adverse consequences, such as liver fibrosis, cirrhosis, hepatocellular carcinoma, end-stage liver disease, and a need for a liver transplant. NASH is discussed in greater detail in Sheka et al., “Nonalcoholic Steatohepatitis: A Review,” JAMA Review, 323(12): 1175-83 (2020), which is herein incorporated by reference in its entirety.

Therapeutic advances in NAFLD progression and management now highlight the role of inflammatory pathway activation (Schuster et al., “Triggering and resolution of inflammation in NASH,” Nat Rev Gastroenterol Hepatol, 15:349-364 (2018)) in NASH development, hepatic fibrogenesis and progression to hepatocellular carcinoma (HCC). Utilizing systems biology and genomic strategies, the present inventors have identified extracellular nicotinamide phosphoribosyltransferase (eNAMPT) (Ye et al., “Pre-B-cell colony-enhancing factor as a potential novel biomarker in acute lung injury,” Am J Respir Crit Care Med, 171:361-370 (2005)) as a novel damage-associated molecular pattern protein (DAMP) that potently regulates innate immunity-mediated inflammation and fibrosis via ligation of TLR4 (Camp et al., “Unique Toll-Like Receptor 4 Activation by NAMPT/PBEF Induces NFkappaB Signaling and Inflammatory Lung Injury,” Scientific Reports, 5:13135 (2015); Oita et al., “Novel Mechanism for Nicotinamide Phosphoribosyltransferase Inhibition of TNF-alpha-mediated Apoptosis in Human Lung Endothelial Cells,” Am J Respir Cell Mol Biol, 59:36-44 (2018)). NAMPT plays an essential role in multi-organ inflammation, injury and fibrosis (lung, liver, renal, cardiac) and in cancer (Sun et al., “Role of secreted extracellular nicotinamide phosphoribosyltransferase (eNAMPT) in prostate cancer progression: Novel biomarker and therapeutic target,” EBioMedicine, 61:103059 (2020)) (FIG. 1). The same NAMPT protein, when intracellular as iNAMPT, enzymatically regulates NAD metabolism, and has been targeted with small-molecule inhibitors (SMI) in clinical cancer trials, but failed due to lack of benefit and toxicity (Sun et al., “Role of secreted extracellular nicotinamide phosphoribosyltransferase (eNAMPT) in prostate cancer progression: Novel biomarker and therapeutic target,” EBioMedicine, 61:103059 (2020)). The present disclosure shows NAMPT (e.g., eNAMPT) as a key regulator of NAFLD progression to NASH and as an attractive therapeutic target to reduce NAFLD progression to later stages. As described in the “Examples” Section, increased hepatic NAMPT expression is seen in murine NAFLD/NASH models and in humans with NAFLD. Furthermore, a NAMPT-neutralizing mAb (ALT-100 mAb) reduced multiple indices of NASH severity, suggesting ALT-100 mAb to be a viable and novel approach to address the urgent unmet need to prevent NASH progression/lethality.

Renal fibrosis, characterized by tubulointerstitial fibrosis and glomerulosclerosis, is the final manifestation of chronic kidney disease. Renal fibrosis is characterized by an excessive accumulation and deposition of extracellular matrix components. This pathologic result usually originates from both underlying complicated cellular activities such as epithelial-to-mesenchymal transition, fibroblast activation, monocyte/macrophage infiltration, and cellular apoptosis and the activation of signaling molecules such as transforming growth factor beta and angiotensin II. However, because the pathogenesis of renal fibrosis is extremely complicated and knowledge regarding this condition is still limited, further studies are needed. Renal fibrosis is discussed in greater detail in Nogueira et al., “Pathophysiological Mechanisms of Renal Fibrosis: A Review of Animal Models and Therapeutic Strategies,” In Vivo, 31(1): 1-22 (2017); Liu, “Cellular and Molecular Mechanisms of Renal Fibrosis,” Nat Rev Nephrol, 7(12): 684-696 (2011); Arai and Yanagita, “Janus-Faced: Molecular Mechanisms and Versatile Nature of Renal Fibrosis,” Kidney360, 1(7): 697-704 (2020); Meng et al., “Inflammatory processes in renal fibrosis,” Nat Rev Nephrol, 10:493-503 (2014); and Boor et al., “Treatment targets in renal fibrosis,” Nephrol Dial Transplant, 22:3391-3407 (2007), each of which are incorporated herein by reference in their entireties.

### Biomarkers, Single Nucleotide Polymorphisms, and Uses Thereof

Provided are methods for identifying a subject having or at risk of developing an inflammatory condition, such as cardiac ischemia, traumatic brain injury, cancer, chorioamnionitis, NASH, or renal fibrosis. Some embodiments comprise methods of diagnosing an inflammatory condition. Some embodiments comprise determining progression of an inflammatory condition. Some embodiments comprise determining efficacy of an inflammatory condition treatment.

Some embodiments comprise detecting a presence or absence of NAMPT in a sample. Some embodiments comprise detecting a level of NAMPT in a sample. In some embodiments, a subject is determined to have, or be at risk of developing, an inflammatory condition based on the presence or level (e.g., increased level) of NAMPT in the sample. In some embodiments, the subject is determined not to have, or not to be at risk of developing, an inflammatory condition based on or based at least in part on the absence of or a low or decreased level of NAMPT in the sample.

Some embodiments comprise detecting a presence, absence, or level of one or more additional biomarkers, such as cytokine chemokines (e.g., IL-6, IL-8, IL-1b, and/or IL-RA); dual functioning enzymes such as macrophage migration inhibitory factor); vascular injury markers (e.g., VEGRA, S1PR3, and/or angiopoietin 2); and/or advanced glycosylation end product pathway markers (e.g., HMGB1 and/or soluble RAGE). Some embodiments comprise determining an increased or decreased risk that a subject has or will develop an inflammatory condition based on or based at least in part on the level of one or more of the preceding markers (e.g., in combination with the presence, absence, or level of NAMPT).

Single nucleotide polymorphisms (SNPs) are located in gene promoters, exons, introns as well as 5′- and 3′-untranslated regions (UTRs) and affect gene expression by different mechanisms. Provided are SNPs located in the promoter region of the human NAMPT gene.

In some embodiments, one or more of the following SNPs are associated with an inflammatory condition: rs7789066 (position: chr7:106287306 (GRCh38.p12)); rs116647506 (position: chr7:106287180 (GRCh38.p12)); rs61330082 (position: chr7:106286419 (GRCh38.p12)); rs114382471 (position: chr7:106286288 (GRCh38.p12)); rs9770242 (position: chr7:106285885 (GRCh38.p12)); rs59744560 (position: chr7:106285832 (GRCh38.p12)); rs190893183 (position: chr7:106285663 (GRCh38.p12)); and rs1319501 (position: chr7:106285307 (GRCh38.p12)).

Some embodiments comprise detecting 2, 3, 4, 5, 6, 7, or 8 SNPs selected from the group consisting of rs7789066; rs116647506; rs61330082; rs114382471; rs9770242; rs59744560; rs190893183; and rs1319501.

In some embodiments, a SNP used in the methods described herein is rs7789066, rs61330082, rs9770242, and/or rs59744560. In an alternative embodiment, a SNP used in the methods described herein is rs116647506, rs114382471, rs190893183, and/or rs1319501.

Without being bound by theory, it is believed the SNPs described herein may contribute to dysregulation of cellular processes including dysregulation of inflammatory signaling pathways (e.g., NFkB-dependent inflammatory cascades) and lead to the progression of an inflammatory condition. It is contemplated that the SNPs disclosed herein, that occur within the promoter region of human NAMPT, cause increased NAMPT promoter activity. The increased activity leads to an increased expression of NAMPT and subsequently, increased plasma levels of NAMPT. The possible increase in the levels of NAMPT might activate the evolutionarily-conserved, NFkB-dependent inflammatory cascades via Toll-like receptor 4 (TLR4). The enhanced production of cytokines in turn enhance an inflammatory condition phenotype, thus increasing the risk of a subject developing the condition. NAMPT also influences the microenvironment surrounding cells impacted by inflammatory conditions, thus influencing disease development and/or progression.

Some embodiments comprise obtaining a sample from a subject at risk of having or developing an inflammatory condition and detecting the presence or absence of one or more SNPs associated with the condition. In some embodiments, the subject at risk of having or developing an inflammatory condition exhibits one or more symptoms of the inflammatory condition. Such symptoms for inflammatory conditions, including symptoms that may be specific to one or more inflammatory conditions such as cardiac ischemia, traumatic brain injury, cancer, chorioamnionitis, NASH, and renal fibrosis are well known in the art. In some embodiments, the subject is asymptomatic but has one or more risk factors for developing the inflammatory condition. Such risk factors for inflammatory conditions, including risk factors that may be specific to one or more inflammatory conditions such as cardiac ischemia, traumatic brain injury, cancer, chorioamnionitis, NASH, and renal fibrosis are well known in the art. In some embodiments, the presence of at least one SNP in the sample indicates that the subject has or is at risk for developing the inflammatory condition. In some embodiments, the presence of 2, 3, 4, 5, 6, 7, or 8 SNPs selected from the group consisting of rs7789066; rs116647506; rs61330082; rs114382471; rs9770242; rs59744560; rs190893183; and rs1319501 indicates the subject has or is at risk of developing the inflammatory condition. Some embodiments comprise diagnosing a subject as having the inflammatory condition based on the presence of one or more SNPs. In some embodiments, the presence of 1, 2, 3, 4, 5, 6, 7, or 8 SNPs selected from the group consisting of rs7789066; rs116647506; rs61330082; rs114382471; rs9770242; rs59744560; rs190893183; and rs1319501 indicates that the subject is likely to be responsive or benefit from treatment with an NAMPT inhibitor as described elsewhere herein. Those skilled in the art would be able to determine which one or more inflammatory conditions the subject has or is at risk of developing based on the collection and evaluation of routine clinical information that is known in the art. The presence of one or more SNPs may be used to strengthen or corroborate a diagnosis.

In some embodiments, detection of at least one SNP in the promoter element of NAMPT from a sample can be achieved by SNP genotyping. Generally, SNP genotyping includes steps of, for example, collecting a biological sample from a test subject (e.g., sample of biopsied tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). SNP genotyping can identify SNPS that are either homozygous or heterozygous. In some embodiments of the method described herein, the at least one SNP is homozygous. In other embodiments, at least one SNP is heterozygous.

Other methods of detecting SNPs are known to the art and can be applied to the present methods. For example, an assay system that is commercially available and can be used to identify a nucleotide occurrence of one or more SNPs is the SNP-IT™ assay system (Orchid BioSciences, Inc.; Princeton N.J.). In general, the SNP-IT™ method is a three step primer extension reaction. In the first step a target nucleic acid molecule is isolated from a sample by hybridization to a capture primer, which provides a first level of specificity. In a second step the capture primer is extended from a terminating nucleotide triphosphate at the target SNP site, which provides a second level of specificity. In a third step, the extended nucleotide triphosphate can be detected using a variety of known formats, including, for example, by direct fluorescence, indirect fluorescence, an indirect colorimetric assay, mass spectrometry, or fluorescence polarization. Reactions conveniently can be processed in 384 well format in an automated format using a SNPSTREAM™ instrument (Orchid BioSciences, Inc.).

Nucleic acid samples from a sample taken from a subject can be genotyped to determine the presence and identity of a SNP of interest by methods known to a person of skill in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues Mol. Biol. 2003 April; 5(2):43-60; Shi, “Technologies for individual genotyping: detection of genetic polymorphisms in drug targets and disease genes”, Am J Pharmacogenomics. 2002; 2(3): 197-205; and Kwok, “Methods for genotyping single nucleotide polymorphisms”, Annu Rev Genomics Hum Genet 2001; 2:235-58. Exemplary techniques for high-throughput SNP genotyping are described in Marnellos, “High-throughput SNP analysis for genetic association studies”, Curr Opin Drug Discov Devel. 2003 May; 6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection. Various methods for detecting polymorphisms include, but are not limited to, methods in which protection from cleavage agents is used to detect mismatched bases in RNA/RNA or RNA/DNA duplexes (Myers et al, Science 230: 1242 (1985); Cotton et al, PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285: 125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and SI protection or chemical cleavage methods.

In some embodiments, detecting a SNP in the NAMPT promoter sequence comprises contacting a sample from a subject with an oligonucleotide probe that selectively hybridizes to a nucleotide sequence comprising the SNP, or a nucleotide sequence complementary thereto, and detecting selective hybridization of the oligonucleotide probe. In certain embodiments, an oligonucleotide probe that selectively hybridizes to a nucleotide sequence comprising a SNP includes 100-500 (e.g., 100, 125, 150, 175, 200, 225, 250, 275, 300, 325, 350, 375, 400, 425, 450, 475, or 500) base pairs on each side surrounding the SNP. For example, an oligonucleotide probe that selectively hybridizes to a nucleotide sequence comprising a SNP can include 200 base pairs on each side surrounding the SNP. In particular embodiments, an oligonucleotide probe comprising the nucleotide sequence set forth in SEQ ID NO: 18 selectively hybridizes to a nucleotide sequence comprising rs7789066; an oligonucleotide probe comprising the nucleotide sequence set forth in SEQ ID NO: 19 selectively hybridizes to a nucleotide sequence comprising rs61330082; an oligonucleotide probe comprising the nucleotide sequence set forth in SEQ ID NO: 20 selectively hybridizes to a nucleotide sequence comprising rs9770242; an oligonucleotide probe comprising the nucleotide sequence set forth in SEQ ID NO: 21 selectively hybridizes to a nucleotide sequence comprising rs59744560; and/or an oligonucleotide probe comprising the nucleotide sequence set forth in SEQ ID NO: 22 selectively hybridizes to a nucleotide sequence comprising rs1319501. Exemplary oligonucleotide probes that can selectively hybridize to nucleotide sequences comprising the SNPs are provided in Table 1 below:

TABLE 1
Exemplary oligonucleotide probes for detecting SNPs

| | | Sequence |
|---|---|---|
| Description | Sequence | identifier |
| probe for | AATGTGGGCTTTGTTTATGGTAGTATTTTTTTAAGA | SEQ ID |
| detecting | TGCAAAATTTGATCTTGCAATCTTTGAGTTGAATTT | NO: 18 |
| rs7789066 | GCAGTTTTAAAATAAAAAGGTCTTATATCTGTGCAA | |
| (SNP | AGAAAAAATATTGTATTGACATTGCTTGTTAAATTA | |
| underlined) | AGGAGTGAGGCCTGCACAAGTATTAGTAATGTGAAT | |
| | CCTCACAGTAGTCTCCAGAGAAAAAAAATGACAATG | |
| | AAGTCATGTTACCAATAGGACAATCACCATTTGCCT | |
| | GAGATAGAAATAGGCACATTCTCTATGTAACTACAT | |
| | GCTTAAGCTGGAGCAATTCAGAATTAATTGGGGTTT | |
| | AGAACTATGAAATTATCACTGAAAACAGAGCCAAGA | |
| | TTTCATTTTAAAATGGCCTCCCCTGAAAGACAGTTT | |
| | AACAG | |
| probe for | AGTGGAACTTGTGAATTGAGATTCATAGTGGAACTT | SEQ ID |
| detecting | GTGAATTGAGATTCATCTCGAAACTGGAGGCATGGC | NO: 19 |
| rs61330082 | TGAGACTTCTAATAAAGACAACCTCAGTCAACACTA | |
| (SNP | TGTCTTGAAGTCAGTATATATTTTTGACAATCACCT | |
| underlined) | CATCTACACGTAGATACAATACAGGGCAAAGATCAT | |
| | GGAAGTGGAAGGTATCACCAGGCACTCACCAATGTA | |
| | GTAAATACTAGTACACTTACAATTATTTTCAGCAAC | |
| | GAGGTTTGAAACAAGAGGGCTTATGTATTTATTGGT | |
| | TGATCTTCCCTGTGTTTTACCGGGGAAAATTATTTG | |
| | TAAACGCATTTAAACAAATTATTATTTCTATTTTGA | |
| | GACGGAGTCTCGCTCTGTCGCCCAGGCTGGAGTGCA | |
| | GTGGC | |
| probe for | CCGCTTTCCTCCGGCGGCTCTGTCTATGGCTGAGCT | SEQ ID |
| detecting | CTTTGATCCTTTGAGAGATGGTTTGACTTTTCCCGA | NO: 20 |
| rs9770242 | GCAAAGAGCCTGCGTTGAAAAGCGGGGGTGGAATTC | |
| (SNP | AGTCCTCACAGATAATGAGGGGACAAGACCTAATTG | |
| underlined) | AACCGAGTATTGCCGGGAAGGAAAAGGCAACGGGCC | |
| | AAGCCTTTGACAGGGTGCGACACTGACTTTTATCAT | |
| | CGTTATAGTCTTTAAATCCTGGGAAACGAGTTGGCA | |
| | ACCCCAAAATAAAGAAGTGTAATGACGTCTGATGAC | |
| | TTCACCCAAATACAGACCATTCCAAGAAAGACTTGC | |
| | GCAGTTCTCATGCGTGGTTGCGTTTTTGCATAAAAC | |
| | TAAGATTCCCTTTGTCCGCATGTTTAATAGCTTAAA | |
| | AATAA | |
| probe for | GAGCTGCGGTGAGGAGTGAGGCTGAGGGGCCCCTTT | SEQ ID |
| detecting | CATCTGATGCAGCGACTCCGCTTTCCTCCGGCGGCT | NO: 21 |
| rs59744560 | CTGTCTATGGCTGAGCTCTTTGATCCTTTGAGAGAT | |
| (SNP | GGTTTGACTTTTCCCGAGCAAAGAGCCTGCGTTGAA | |
| underlined) | AAGCGGGGGTGGAATTCAGTCCTCACAGATAATGAG | |
| | GGGACAAGACCTAATTGAACCGAGTATTGCCGGGAA | |
| | GGAAAAGGCAACGGGCCAAGCCTTTGACAGGGTGCG | |
| | ACACTGACTTTTATCATCGTTATAGTCTTTAAATCC | |
| | TGGGAAACGAGTTGGCAACCCCAAAATAAAGAAGTG | |
| | TAATGACGTCTGATGACTTCACCCAAATACAGACCA | |
| | TTCCAAGAAAGACTTGCGCAGTTCTCATGCGTGGTT | |
| | GCGTT | |
| probe for | GGGAGCTCTGGCGGACTCCCCACCTCGGTTCCCCCG | SEQ ID |
| detecting | CCTTCACCCCGTCACCCTCCGGGGGCCGAGAAAGGG | NO: 22 |
| rs1319501 | CGGGGCGCGGCAGCGCGCTGCGCAGTGCGCGGAGGC | |
| (SNP | GGGGCGGGGAGGAGGACGTGATGCACGCGCTCTTCC | |
| underlined) | TCCCAGACGCCAGCTCTGGGAAGCTGGAGGCAGCGG | |
| | GGCAGCCCCGGCGCGTGACCCGGGCGCTTACCTAAG | |
| | TTCGAGTTCCCGGCACGGGCGCGGGAGGGCGGGGCC | |
| | TGGAGGGGGCGTTCCCAGCTTTGCCAGTGCCACGAG | |
| | GAGCCGGTTCGCCCGCCCCGCCTGGGACCTTCCGTC | |
| | CTACCCAGTCCTGGCCGGTTTTCTGGGTCCTCCTGA | |
| | AGTCACGCCACCCGGCTAGGGGGCGAGGAGCCTCCT | |
| | ACTGC | |

In some embodiments, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced. During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present. In some embodiments of the method, the oligonucleotide comprises a bilabeled oligonucleotide probe, comprising a fluorescent moiety and a fluorescent quencher.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in prognostic assays for a variety of inflammatory conditions, including cardiac ischemia, traumatic brain injury, cancer, chorioamnionitis, NASH and renal fibrosis, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

The SNPs may also be detected using a mismatch detection technique, including but not limited to the RNase protection method using riboprobes (Winter et al, Proc. Natl. Acad Sci. USA 82:7575, 1985; Meyers et al, Science 230:1242, 1985) and proteins which recognize nucleotide mismatches, such as the E. coli mutS protein (Modrich, P. Ann. Rev. Genet. 25:229-253, 1991). Alternatively, SNPs can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nuci. Acids Res. 18:2699-2706, 1990; Sheffield et al., Proc. Nat. Acad. Sci. USA 86:232-236, 1989).

In some embodiments, a SNP described herein can be detected using a method based on mass spectrometry. Mass spectrometry takes advantage of the unique mass of each of the four nucleotides of DNA. SNPs can be unambiguously detected by mass spectrometry by measuring the differences in the mass of nucleic acids having SNP compared to the samples from the control subject lacking SNPs. MALDI-TOF (Matrix Assisted Laser Desorption Ionization—Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

SNP genotyping is useful for numerous applications, including, but are not limited to, SNP-disease association analysis, disease predisposition screening, disease diagnosis, disease prognosis, disease progression monitoring, determining therapeutic strategies based on an individual's genotype, developing effective therapeutic agents (e.g., an anti-NAMPT antibody) based on SNP genotypes associated with a disease or likelihood of responding to a drug, and stratifying a patient population for clinical trial for a treatment regimen.

### Methods of Treatment

Also provided are methods of treating a subject having or at risk of developing an inflammatory condition, such as cardiac ischemia, traumatic brain injury, cancer, chorioamnionitis, NASH, or renal fibrosis. Some embodiments comprise identifying a subject having or at risk for developing the inflammatory condition, and treating the subject so as to prevent or reduce the development or progression of the condition. Some embodiments comprise preventing an adverse consequence of an inflammatory condition by treating a subject having or at risk of developing the condition.

In some embodiments, the subject is treated by administering a NAMPT inhibitor to the subject (e.g., to reduce levels of NAMPT and/or reduce NAMPT activity). In some embodiments, a NAMPT inhibitor is an anti-NAMPT antibody. In some embodiments, the anti-NAMPT antibody is an antibody as described in U.S. Pat. App. Pub. No. 2021/0070883 to Garcia et al., published on Mar. 11, 2021, which is herein incorporated by reference in its entirety. Exemplary anti-NAMPT antibodies comprise Ab1, Ab2, and Ab3, or antigen binding portions thereof, as described in Table 2 below.

TABLE 2
Exemplary anti-NAMPT antibodies

| | | | Sequence |
|---|---|---|---|
| Description | | Sequence | identifier |
| AB 1 | AB 1 heavy chain | QVQLVQSGAEVTKPGASVKVSCKASGY | SEQ ID NO: 2 |
| | variable region | TFTSYWMQWVRQAPGQGLEWVGEIDPS | |
| | (VH) | NSYTNYNQKFRGRVTLTRDTSTTTVYM | |
| | (CDRs | ELSSLRSEDTAVYYCARGGYWGQGTTV | |
| | underlined) | TVSS | |
| | AB 1 light chain | DIVMTQSPLSLPVTPGEPASISCRSSK | SEQ ID NO: 3 |
| | variable region | SLLHSQGITYLYWYLQKPGQSPQLLIY | |
| | (VL) | QLSNRASGVPDRFSGSGSGTDFTLKIS | |
| | (CDRs | RVEAEDVGVYYCVQNLELPYTFGGGTK | |
| | underlined) | LEIK | |
| | AB 1 CDR-H1 | GYTFTSYWMQ | SEQ ID NO: 4 |
| | AB 1 CDR-H2 | EIDPSNSYTNYNQKFRG | SEQ ID NO: 5 |
| | AB 1 CDR-H3 | ARGGY | SEQ ID NO: 6 |
| | AB 1 CDR-L1 | RSSKSLLHSQGITYLY | SEQ ID NO: 7 |
| | AB 1 CDR-L2 | QLSNRAS | SEQ ID NO: 8 |
| | AB 1 CDR-L3 | VQNLELPYT | SEQ ID NO: 9 |
| AB 2 | AB 2 heavy chain | EVQLVQSGAEVKKPGESLRISCKASGY | SEQ ID NO: 10 |
| | variable region | TFTSYWMHWVRQMPGKGLEWMGEIDPS | |
| | (VH) | DSYTNYNQKFKGHVTISADKSISTAYL | |
| | (CDRs | QWSSLKASDTAMYYCAKSNYVVPWYFD | |
| | underlined) | VWGQGTLVTVSS | |
| | AB 2 light chain | EIVLTQSPGTLSLSPGERATLSCRSSK | SEQ ID NO: 11 |
| | variable region | SLLHSNGITYLYWYQQKPGQAPRLLIY | |
| | (VL) | QMSNLASGIPDRFSGSGSGTDFTLTIS | |
| | (CDRs | RLEPEDFAVYYCAQNLELPWTFGGGTK | |
| | underlined) | LEIK | |
| | AB 2 CDR-H1 | GYTFTSYWMH | SEQ ID NO: 12 |
| | AB 2 CDR-H2 | EIDPSDSYTNYNQKFKG | SEQ ID NO: 13 |
| | AB 2 CDR-H3 | AKSNYVVPWYFDV | SEQ ID NO: 14 |
| | AB 2 CDR-L1 | RSSKSLLHSNGITYLY | SEQ ID NO: 15 |
| | AB 2 CDR-L2 | QMSNLAS | SEQ ID NO: 16 |
| | AB 2 CDR-L3 | AQNLELPWT | SEQ ID NO: 17 |
| AB 3 | AB 3 heavy chain | QVQLVQSGAEVTKPGASVKVSCKASGY | SEQ ID NO: 23 |
| | variable region | TFTSYWMQWVRQAPGQGLEWVGEIEPS | |
| | (VH) | NSYTNYNQKFRGRVTLTRDTSTTTVYM | |
| | (CDRs | ELSSLRSEDTAVYYCARGGYWGQGTTV | |
| | underlined) | TVSS | |
| AB 3 | light chain | DIVMTQSPLSLPVTPGEPASISCRSSK | SEQ ID NO: 3 |
| | variable region | SLLHSQGITYLYWYVQKPGQSPQLLIY | |
| | (VL) | QLSNRASGVPDRFSGSGSGTDFTLKIS | |
| | (CDRs | RVEAEDVGVYYCVQNLELPYTFGGGTK | |
| | underlined) | LEIK | |
| | AB 3 CDR-H1 | GYTFTSYWMQ | SEQ ID NO: 4 |
| | AB 3 CDR-H2 | EIEPSNSYTNYNQKFRG | SEQ ID NO: 24 |
| | AB 3 CDR-H3 | ARGGY | SEQ ID NO: 6 |
| | AB 3 CDR-L1 | RSSKSLLHSQGITYLY | SEQ ID NO: 7 |
| | AB 3 CDR-L2 | QLSNRAS | SEQ ID NO: 8 |
| | AB 3 CDR-L3 | VQNLELPYT | SEQ ID NO: 9 |

Some embodiments comprise administering, to a subject having or at risk for cardiac ischemia, one or more of aspirin, nitrates, beta blockers, calcium channel blockers, cholesterol-lowering drugs, angiotensin-converting enzyme (ACE) inhibitors, and ranolazine. Some embodiments comprise administering one or more of the above therapies in combination with one or more NAMPT inhibitors (e.g., antibodies).

Some embodiments comprise administering, to a subject having or at risk for myocardial infarction, one or more of aspirin, thrombolytics, antiplatelet agents, blood thinning medications, pain relievers, nitroglycerin, beta blockers, ACR inibitors, and statins. Some embodiments comprise administering one or more of the above therapies in combination with one or more NAMPT inhibitors (e.g., antibodies).

Some embodiments comprise performing surgery on (e.g., to remove hematomas, repair skull fractures, stop brain bleeding, or open a window in the skull to reduce pressure inside the skull) and/or administering, to a subject having or at risk for traumatic brain injury, one or more of oxygen, blood, diuretics, anti-seizure drugs, and coma-inducing drugs. Some embodiments comprise administering one or more of the above therapies in combination with one or more NAMPT inhibitors (e.g., antibodies).

Some embodiments comprise administering, to a subject having or at risk for cancer, one or more of an alkylating agent, an antimetabolite, an antimitotic, a cytotoxic antibiotic, a polyamine inhibitor, an iron-modulating drug, a chemotherapeutic agent, a kinase inhibitor, a monoclonal antibody, a tyrosine kinase inhibitor, a serine/threonine-protein kinase inhibitor, an immune checkpoint inhibitor, a radiotherapy, and a chimeric antigen receptor T cell (CAR-T). Exemplary therapies are set forth in Falzone et all., “Evolution of Cancer Pharmacological Treatments at the Turn of the Third Millennium,” Front. Pharmacol., 9 (Article 1300): 1-26 (2018), which is incorporated herein by reference in its entirety. Some embodiments comprise administering one or more of the above therapies in combination with one or more NAMPT inhibitors (e.g., antibodies).

Some embodiments comprise administering one or more antibiotics to a subject having or at risk of developing chorioamnionitis. Exemplary antibiotics include ampicillin, penicillin, gentamicin, clindamycin, cefazolin, vancomycin, and metronidazole. In some embodiments, the antibiotic(s) is administered intravenously. In some embodiments, the antibiotic is administered orally. Some embodiments comprise administering one or more antibiotics in combination with one or more NAMPT inhibitors (e.g., antibodies).

Some embodiments comprise administering one or more of an antioxidant (e.g., vitamin E), a PPAR-γ agonist (e.g., a thiazolidinedione such as pioglitazone), and a glucagon-like peptide 1 (GLP-1) agonist to a subject having or at risk of developing NASH. Some embodiments comprise administering one or more of the antioxidant, PPAR-γ agonist, and GLP-1 agonist in combination with one or more NAMPT inhibitors (e.g., antibodies).

Some embodiments comprise administering one or more of angiotensin-converting enzyme inhibitors (ACEI), angiotensin II receptor type 1 blockers (ARB), kallikrein gene therapy (e.g., adenoviral delivery of human tissue kallikrein gene), human recombinant relaxin, IL-1 receptor antagonist, BMP-7, Anti-TNF antibody, CCR1 antagonist, anti-PDGF-D antibody, and VEGF121 to a subject having or at risk of developing renal fibrosis. Such therapeutics can be administered intravenously, orally, or via adenoviral vectors. Some embodiments comprise administering one or more of ACEI, ARB, kallikrein gene therapy, human recombinant relaxin, IL-1 receptor antagonist, BMP-7, Anti-TNF antibody, CCR1 antagonist, anti-PDGF-D antibody, and VEGF121 in combination with one or more NAMPT inhibitors (e.g., antibodies).

Any of the aspects or embodiments disclosed herein may be combined with each other unless clearly not feasible or practical from context. For example, any feature of one embodiment may be added to the features of another embodiment or may replace a corresponding feature of another embodiment. The below Examples further describe and demonstrate the compositions and methods of the present disclosure. The Examples are not intended to limit the disclosure in any way. Other aspects will be apparent to those skilled in the art. For example, in each instance herein any of the terms “comprising”, “consisting essentially of” and “consisting of” may be replaced with either of the other two terms; moreover, any of the terms may be used in reference to features disclosed herein.

### EXAMPLES

### Example 1: Identification of SNPs Associated with Inflammatory Conditions

NAMPT promoter SNPs have been identified as indicators that may be used to identify patients having or having an increased risk for an inflammatory condition, such as cardiac ischemia, traumatic brain injury, cancer, chorioamnionitis, nonalcoholic steatohepatitis (NASH), or renal fibrosis.

NAMPT SNPs were reviewed and refined for assessing risk for inflammatory conditions, with several significantly over-represented in African descent individuals. The SNPs are, rs7789066 (position: chr7:106287306 (GRCh38.p12)), rs116647506 (position: chr7:106287180 (GRCh38.p12)), rs61330082 (position: chr7:106286419 (GRCh38.p12)), rs114382471 (position: chr7:106286288 (GRCh38.p12)), rs9770242 (position: chr7:106285885 (GRCh38.p12)), rs59744560 (position: chr7:106285832 (GRCh38.p12)), rs190893183 (position: chr7:106285663 (GRCh38.p12)), and rs1319501 (position: chr7:106285307 (GRCh38.p12)).

These NAMPT SNPs contribute to inflammatory condition susceptibility and altering NAMPT promoter activity in response to mechanical stress and to hypoxia with key involvement by hypoxia-induced transcription factor HIF2α significantly influenced by NAMPT promoter SNPs-948T, -1001G, and -2422G, but not by -1535G, known to be a protective SNP in some situations.

### Example 2: NAMPT SNPs and/or NAMPT Expression for Identifying Subjects Having or at Risk of Developing Cardiac Ischemia

The present example describes how NAMPT SNPs and/or NAMPT expression level could be used for identification of subjects having cardiac ischemia or at risk of developing cardiac ischemia. These methods can be useful for diagnosing the presence of cardiac ischemia or the risk of developing cardiac ischemia in subjects who exhibit one or more symptoms of the disease, or in asymptomatic subjects with one or more risk factors for developing the disease. Biological samples obtained from such subjects can be assessed for presence of NAMPT SNPs and/or evaluated for NAMPT expression levels.

For example, biological samples obtained from such subjects can be assessed for presence of at least one NAMPT SNP described herein (e.g., presence of 1, 2, 3, 4, 5, 6, 7, or 8 SNPs selected from the group consisting of rs7789066; rs116647506; rs61330082; rs114382471; rs9770242; rs59744560; rs190893183; and rs1319501). Detection of at least one SNP in the promoter element of NAMPT from a biological sample can be achieved by methods described hereinabove, such as by SNP genotyping. SNP genotyping can identify SNPS that are either homozygous or heterozygous. The presence of at least one SNP in biological sample from a subject may indicate that the subject has or is at risk for developing cardiac ischemia. Moreover, presence of at least one SNP in biological sample from a subject may also identify the subject as one who would be more responsive to treatment of cardiac ischemia using a NAMPT inhibitor, such as a neutralizing anti-NAMPT antibody (Ab). In some instances, absence of the aforementioned SNPs in biological sample from a subject may indicate that the subject does not have cardiac ischemia or is at low risk for developing cardiac ischemia.

Additionally, or alternatively, biological samples obtained from such subjects can be evaluated for NAMPT protein expression levels. Expression level of NAMPT in biological sample from a subject (e.g., a test subject) can be evaluated by various methods, including, but not limited to, immunohistochemistry (IHC), western blot analyses, ELISA, immunoprecipitation, autoradiography, antibody array, and real-time quantitative reverse transcription PCR (Real-Time qRT-PCR or RT-qPCR). Expression level of NAMPT, as evaluated in biological sample from a test subject, can be compared to a standard control, such as NAMPT expression level in healthy controls (e.g., subjects without cardiac disease and/or subjects without inflammatory disease). Increased NAMPT expression level in a test subject compared to standard control may indicate that the subject has or is at risk for developing cardiac ischemia. Moreover, increased NAMPT expression level in biological sample from a subject may also identify the subject as one who would be more responsive to treatment of cardiac ischemia using a NAMPT inhibitor, such as a neutralizing anti-NAMPT Ab. In some instances, similar or reduced NAMPT expression level in a test subject compared to standard control may indicate that the test subject does not have cardiac ischemia or is at low risk for developing cardiac ischemia.

### Example 3: Anti-NAMPT Ab for Treating and/or Preventing Cardiac Ischemia

The present example describes how a NAMPT inhibitor, such as a neutralizing anti-NAMPT Ab could be used for treating a subject having cardiac ischemia or at risk of developing cardiac ischemia. Such subjects can be identified by the methods described in the foregoing example.

A subject having cardiac ischemia or at risk of developing cardiac ischemia can be treated by administering to the subject a therapeutically effective amount of an anti-NAMPT Ab, such as a humanized anti-NAMPT monoclonal Ab (mAb) described herein. The subject can be treated with the humanized anti-NAMPT mAb alone or in combination with one or more additional therapies, including but not limited to, aspirin, nitrates, beta blockers, calcium channel blockers, cholesterol-lowering drugs, angiotensin-converting enzyme (ACE) inhibitors, and ranolazine. Treatment of the subject with the anti-NAMPT Ab, alone or in combination with one or more of the additional therapies, can prevent or reduce the development of cardiac ischemia, can prevent or reduce the progression of cardiac ischemia, and/or can reduce one or more symptoms associated with cardiac ischemia. Treatment of cardiac ischemia using a humanized anti-NAMPT mAb can be more effective in subjects who have higher NAMPT expression and/or presence of at least one NAMPT SNP, as described in the foregoing example.

### Example 4: NAMPT SNPs and/or NAMPT Expression for Identifying Subjects Having or at Risk of Developing Traumatic Brain Injury

The present example describes how NAMPT SNPs and/or NAMPT expression level could be used for identification of subjects having traumatic brain injury or at risk of developing traumatic brain injury. These methods can be useful for diagnosing the presence of traumatic brain injury or the risk of developing traumatic brain injury in subjects who exhibit one or more symptoms of the disease, or in asymptomatic subjects with one or more risk factors for developing the disease. Biological samples obtained from such subjects can be assessed for presence of NAMPT SNPs and/or evaluated for NAMPT expression levels.

For example, biological samples obtained from such subjects can be assessed for presence of at least one NAMPT SNP described herein (e.g., presence of 1, 2, 3, 4, 5, 6, 7, or 8 SNPs selected from the group consisting of rs7789066; rs116647506; rs61330082; rs114382471; rs9770242; rs59744560; rs190893183; and rs1319501). Detection of at least one SNP in the promoter element of NAMPT from a biological sample can be achieved by methods described hereinabove, such as by SNP genotyping. SNP genotyping can identify SNPS that are either homozygous or heterozygous. The presence of at least one SNP in biological sample from a subject may indicate that the subject has or is at risk for developing traumatic brain injury. Moreover, presence of at least one SNP in biological sample from a subject may also identify the subject as one who would be more responsive to treatment of traumatic brain injury using a NAMPT inhibitor, such as a neutralizing anti-NAMPT Ab. In some instances, absence of the aforementioned SNPs in biological sample from a subject may indicate that the subject does not have traumatic brain injury or is at low risk for developing traumatic brain injury.

Additionally, or alternatively, biological samples obtained from such subjects can be evaluated for NAMPT expression levels. Expression level of NAMPT in biological sample from a subject (e.g., a test subject) can be evaluated by various methods, including, but not limited to, IHC, western blot analyses, ELISA, immunoprecipitation, autoradiography, antibody array, and RT-qPCR. Expression level of NAMPT, as evaluated in biological sample from a test subject, can be compared to a standard control, such as NAMPT expression level in healthy controls (e.g., subjects without trauma, brain disease and/or inflammatory disease). Increased NAMPT expression level in a test subject compared to standard control may indicate that the subject has or is at risk for developing traumatic brain injury. Moreover, increased NAMPT expression level in biological sample from a subject may also identify the subject as one who would be more responsive to treatment of traumatic brain injury using a NAMPT inhibitor, such as a neutralizing anti-NAMPT Ab. In some instances, similar or reduced NAMPT expression level in a test subject compared to standard control may indicate that the test subject does not have traumatic brain injury or is at low risk for developing traumatic brain injury.

### Example 5: Anti-NAMPT Ab for Treating and/or Preventing Traumatic Brain Injury

The present example describes how a NAMPT inhibitor, such as a neutralizing anti-NAMPT Ab could be used for treating a subject having traumatic brain injury or at risk of developing traumatic brain injury. Such subjects can be identified by the methods described in the foregoing example.

A subject having traumatic brain injury or at risk of developing traumatic brain injury can be treated by administering to the subject a therapeutically effective amount of an anti-NAMPT Ab, such as a humanized anti-NAMPT mAb described hereinabove. The subject can be treated with the humanized anti-NAMPT mAb alone or in combination with one or more additional therapies, including but not limited to, performing surgery (e.g., to remove hematomas, repair skull fractures, stop brain bleeding, or open a window in the skull to reduce pressure inside the skull) and/or administering one or more of oxygen, blood, diuretics, anti-seizure drugs, and coma-inducing drugs. Treatment of the subject with the anti-NAMPT Ab, alone or in combination with one or more of the additional therapies, can prevent or reduce the development of traumatic brain injury, can prevent or reduce the progression of traumatic brain injury, and/or can reduce one or more symptoms associated with traumatic brain injury. Treatment of traumatic brain injury using a humanized anti-NAMPT mAb can be more effective in subjects who have higher NAMPT expression and/or presence of at least one NAMPT SNP, as described in the foregoing example.

### Example 6: NAMPT SNPs and/or NAMPT Expression for Identifying Subjects Having or at Risk of Developing Cancer

The present example describes how NAMPT SNPs and/or NAMPT expression level could be used for identification of subjects having cancer or at risk of developing cancer. These methods can be useful for diagnosing the presence of cancer or the risk of developing cancer in subjects who exhibit one or more symptoms of the disease, or in asymptomatic subjects with one or more risk factors for developing the disease. Biological samples obtained from such subjects can be assessed for presence of NAMPT SNPs and/or evaluated for NAMPT expression levels.

For example, biological samples obtained from such subjects can be assessed for presence of at least one NAMPT SNP described herein (e.g., presence of 1, 2, 3, 4, 5, 6, 7, or 8 SNPs selected from the group consisting of rs7789066; rs116647506; rs61330082; rs114382471; rs9770242; rs59744560; rs190893183; and rs1319501). Detection of at least one SNP in the promoter element of NAMPT from a biological sample can be achieved by methods described hereinabove, such as by SNP genotyping. SNP genotyping can identify SNPS that are either homozygous or heterozygous. The presence of at least one SNP in biological sample from a subject may indicate that the subject has or is at risk for developing cancer. Moreover, presence of at least one SNP in biological sample from a subject may also identify the subject as one who would be more responsive to treatment of cancer using a NAMPT inhibitor, such as a neutralizing anti-NAMPT antibody (Ab). In some instances, absence of the aforementioned SNPs in biological sample from a subject may indicate that the subject does not have cancer or is at low risk for developing cancer.

Additionally, or alternatively, biological samples obtained from such subjects can be evaluated for NAMPT expression levels. Expression level of NAMPT in biological sample from a subject (e.g., a test subject) can be evaluated by various methods, including, but not limited to, IHC, western blot analyses, ELISA, immunoprecipitation, autoradiography, antibody array, and RT-qPCR. Expression level of NAMPT, as evaluated in biological sample from a test subject, can be compared to a standard control, such as NAMPT expression level in healthy controls (e.g., subjects without cancer and/or subjects without inflammatory disease). Increased NAMPT expression level in a test subject compared to standard control may indicate that the subject has or is at risk for developing cancer. Moreover, increased NAMPT expression level in biological sample from a subject may also identify the subject as one who would be more responsive to treatment of cancer using a NAMPT inhibitor, such as a neutralizing anti-NAMPT Ab. In some instances, similar or reduced NAMPT expression level in a test subject compared to standard control may indicate that the test subject does not have cancer or is at low risk for developing cancer.

### Example 7: Anti-NAMPT Ab for Treating and/or Preventing Cancer

The present example describes how a NAMPT inhibitor, such as a neutralizing anti-NAMPT Ab could be used for treating a subject having cancer or at risk of developing cancer. Such subjects can be identified by the methods described in the foregoing example.

A subject having cancer or at risk of developing cancer can be treated by administering to the subject a therapeutically effective amount of an anti-NAMPT Ab, such as a humanized anti-NAMPT mAb described herein. The subject can be treated with the humanized anti-NAMPT mAb alone or in combination with one or more additional therapies, including but not limited to, an alkylating agent, an antimetabolite, an antimitotic, a cytotoxic antibiotic, a polyamine inhibitor, an iron-modulating drug, a chemotherapeutic agent, a kinase inhibitor, a monoclonal antibody, a tyrosine kinase inhibitor, a serine/threonine-protein kinase inhibitor, an immune checkpoint inhibitor, a radiotherapy, and a chimeric antigen receptor T cell (CAR-T). Treatment of the subject with the anti-NAMPT Ab, alone or in combination with one or more of the additional therapies, can prevent or reduce the development of cancer, can prevent or reduce the progression of cancer, and/or can reduce one or more symptoms associated with cancer. Treatment of cancer using a humanized anti-NAMPT mAb can be more effective in subjects who have higher NAMPT expression and/or presence of at least one NAMPT SNP, as described in the foregoing example.

### Example 8. Assessing NAMPT Expression in Chorioamnionitis

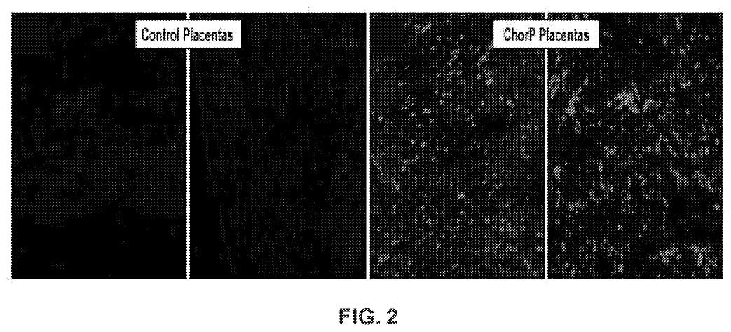

In order to assess the role of NAMPT in chorioamnionitis, expression of NAMPT was assessed by immunohistochemical (IHC) staining in placental tissues obtained from healthy control women or women with chorioamnionitis. As shown in FIG. 2, placental tissues from women with chorioamnionitis (FIG. 2, right panels; “ChorP Placentas”) showed marked increase in NAMPT expression compared to placental tissues from healthy control (FIG. 2, left panels; “Control Placentas”).

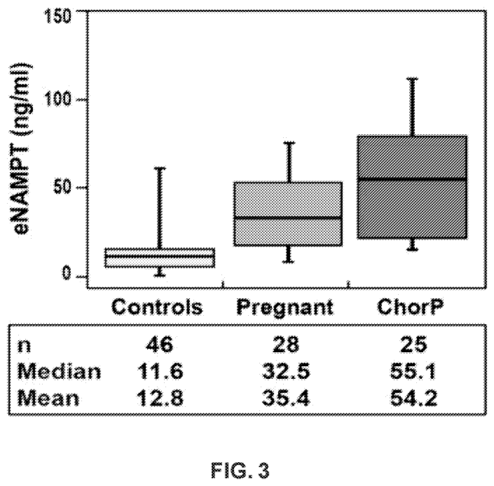

To further assess the role of NAMPT in chorioamnionitis, plasma samples were obtained from healthy pregnant women (“Pregnant”), pregnant women with chorioamnionitis (“ChorP”) or non-pregnant controls (“Control”), and NAMPT plasma levels were assessed by ELISA. The results are described in FIG. 3. As described in FIG. 3, increased NAMPT plasma levels were observed in pregnant women compared to non-pregnant controls, while a marked increase in NAMPT plasma level was observed in pregnant women with chorioamnionitis.

Thus, the results demonstrate a dysregulation of NAMPT expression and secretion in chorioamnionitis, indicating a role of NAMPT in chorioamnionitis pathogenesis.

### Example 9. Validating NAMPT as a Therapeutic Target in Chorioamnionitis Using a Preclinical Model of LPS-Induced Chorioamnionitis

The results outlined in the foregoing example demonstrates a role of NAMPT in chorioamnionitis pathogenesis, thus indicating the potentials of NAMPT as a therapeutic target in chorioamnionitis. Next, to explore the potentials of NAMPT as a therapeutic target in chorioamnionitis, a preclinical murine model of chorioamnionitis and preterm delivery was used, wherein chorioamnionitis was induced by challenging pregnant mice with 20 μg/Kg LPS on day 14 of pregnancy. PBS-challenged pregnant mice were used as controls.

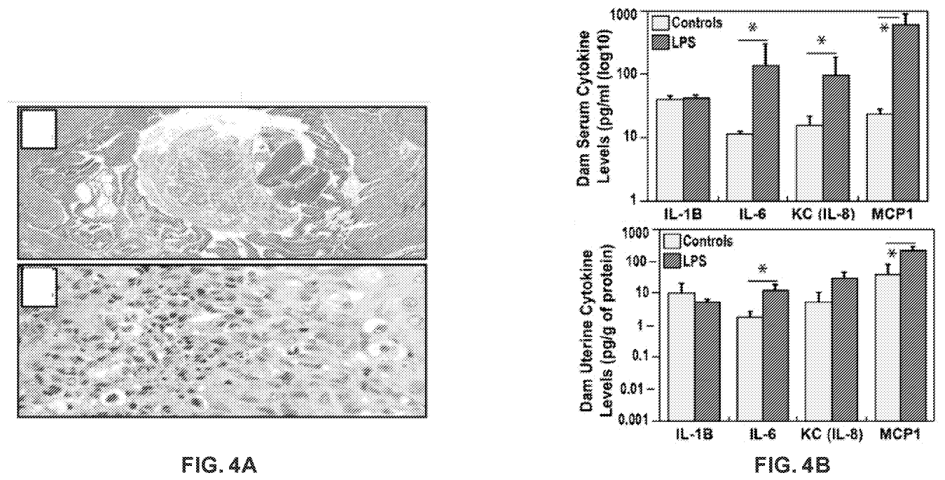

The preclinical model was validated by evaluating the mice for chorioamnionitis-related pathogenesis. To this end, first, uterine tissues from the mice were subjected to hematoxylin and eosin (H&E) staining to assess uterine abscess, inflammation and apoptosis. As shown in FIG. 4A, uterine tissues from the LPS challenged mice showed uterine abscess (FIG. 4A, top panel), and also areas of inflammation and apoptosis (FIG. 4A, bottom panel), thus indicating induction of chorioamnionitis in the preclinical model. To further confirm chorioamnionitis-related pathogenesis in the preclinical model, level of pro-inflammatory cytokines IL-1B, IL-6, KC (IL-8) and MCP1 was evaluated by ELISA in post-partum maternal serum (FIG. 4B, top panel) and uterine tissue homogenate (FIG. 4B, bottom panel) of the LPS-challenged mice. As shown in FIG. 4B, serum levels of IL-6, IL-8 and MCP1 were significantly elevated (p<0.05) in LPS-challenged mice compared to controls (FIG. 4B, top panel), while uterine tissue homogenate from the LPS-challenged mice showed significant increase (p<0.05) in levels of IL-6 and MCP1 compared to their vehicle-challenged counterpart (FIG. 4B, bottom panel). Thus, the results demonstrate chorioamnionitis-related pathogenesis in the LPS-challenged pregnant mice, validating the preclinical model of chorioamnionitis.

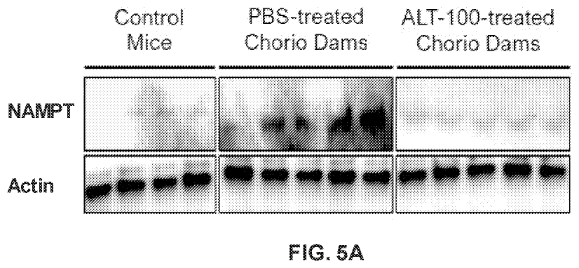

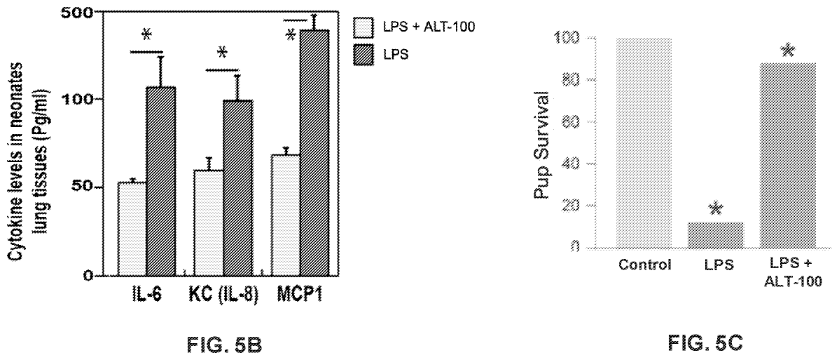

Next, to validate NAMPT as a therapeutic target in chorioamnionitis, pregnant mice from the murine model of chorioamnionitis were treated with 0.4 mg/Kg of a neutralizing anti-NAMPT monoclonal antibody (mAb; “ALT-100” or Ab3 (see, e.g., Table 2 hereinabove)) or vehicle control (PBS). Pups born to the anti-NAMPT mAb treated or PBS-treated chorioamnionitic mice were assessed for percent survival and for expression of NAMPT and other pro-inflammatory cytokines in lung tissues. Pups born to healthy mice served as controls. The results are shown in FIGS. 5A-5C.

As shown in the western blot analysis in FIG. 5A, lung tissue from pups born to PBS-treated chorioamnionitic mice (FIG. 5A, middle panel) showed robust increase in NAMPT expression compared to pups born to healthy controls (FIG. 5A, left panel). In contrast, compared to pups born to PBS-treated chorioamnionitic mice, pups born to anti-NAMPT mAb-treated chorioamnionitic mice (FIG. 5A, right panel) showed a marked decrease in NAMPT expression in lungs. Thus, the results shown in FIG. 5A demonstrate the efficacy of the anti-NAMPT mAb in reducing chorioamnionitis-induced elevation of NAMPT expression in pups. Moreover, as shown in FIG. 5B, lung tissue homogenate from pups born to anti-NAMPT mAb-treated chorioamnionitic mice (“LPS+ALT-100”) showed significant decrease in expression of pro-inflammatory cytokines (IL-6, IL-8 and MCP1) compared to pups born to PBS treated chorioamnionitic mice (p<0.05; “LPS”). Thus, the results shown in FIGS. 5A and 5B demonstrate the efficacy of the anti-NAMPT mAb in reducing chorioamnionitis-related expression of NAMPT and other inflammatory cytokines. Furthermore, as shown in FIG. 5C, pups born to PBS-treated chorioamnionitic mice (“LPS”) showed marked decrease in percent survival (p<0.05) compared to pups born to healthy controls (“Control”). In contrast, compared to pups born to PBS-treated chorioamnionitic mice, pups born to anti-NAMPT mAb-treated chorioamnionitic mice (“LPS+ALT-100”) showed a significant increase in percent survival (p<0.05). Thus, the results shown in FIGS. 5A-5C demonstrate the effectiveness of the anti-NAMPT mAb in reducing inflammation in pups and in increasing survival of pups born to chorioamnionitic females, and validate NAMPT as a therapeutic target in chorioamnionitis.

### Example 10. Validating NAMPT as a Therapeutic Target in Chorioamnionitis Using Chorioamnionitis-Exposed Pups in a Preclinical Model of Bronchopulmonary Dysplasia

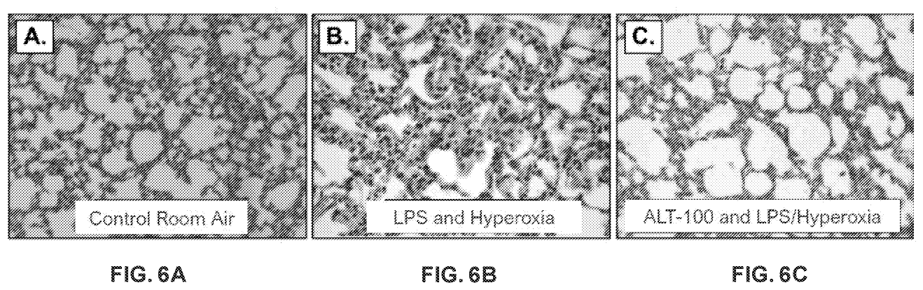

To further validate NAMPT as a therapeutic target in chorioamnionitis, efficacy of anti-NAMPT mAb (“ALT-100”) was tested using chorioamnionitis-exposed pups in a murine model of bronchopulmonary dysplasia (BPD). To this end, pups born to chorioamnionitic females were postnatally exposed to LPS and hyperoxia (FiO2 85%) for 14 days, and then housed in room air (RA) for 10-14 days. The LPS/hyperoxia challenged pups were then treated with neutralizing anti-NAMPT mAb or left untreated. Lung tissues from the mice were subjected to H&E staining to assess inflammation and bronchopulmonary dysplasia 28 days after the LPS/hyperoxia challenge. Lung tissues from pups born to chorioamnionitic females and exposed to control room air served as control. The results are shown in FIGS. 6A-6C.

As shown in FIGS. 6A-6C, lung tissues from LPS/hyperoxia challenged pups (FIG. 6B) showed robust increase in inflammation and bronchopulmonary dysplasia compared to lung tissues from pups that were exposed to control room air (FIG. 6A). In contrast, lung tissues obtained from anti-NAMPT mAb treated mice showed marked reduction in inflammation and bronchopulmonary dysplasia (FIG. 6C). Thus, the results demonstrate the effectiveness of the anti-NAMPT mAb in protecting newborn pups from bronchopulmonary dysplasia and further validate NAMPT as a therapeutic target in chorioamnionitis.

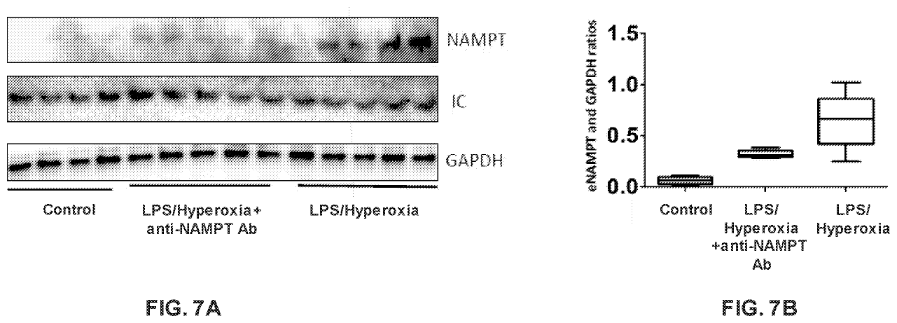

Next, lung tissue homogenates from the mice were subjected to western blot analysis to assess NAMPT expression 28 days after the LPS/hyperoxia challenge. Lung tissues from pups born to chorioamnionitic females and exposed to control room air served as control. The results are shown in FIGS. 7A-7B. As shown in FIGS. 7A-7B, lung tissues from LPS/hyperoxia challenged pups (“LPS/Hyperoxia”) showed robust increase in NAMPT expression compared to lung tissues from pups that were exposed to control room air (“Control”). In contrast, lung tissues obtained from mice that were treated with anti-NAMPT pAb (Bethyl Laboratories) (“LPS/Hyperoxia+anti-NAMPT Ab”) showed marked reduction in NAMPT expression. Thus, the results demonstrate the effectiveness of the anti-NAMPT Ab in reducing NAMPT expression in a bronchopulmonary dysplasia model, and further validate NAMPT as a therapeutic target in chorioamnionitis.

### Example 11. Validating NAMPT as a Therapeutic Target in Chorioamnionitis Using Pre-Clinical Murine Model of Chorioamnionitis-Induced Pulmonary Hypertension

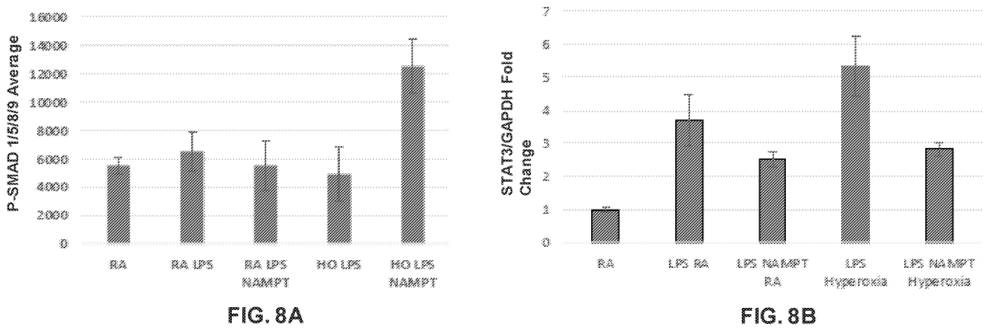

To further validate NAMPT as a therapeutic target in chorioamnionitis, efficacy of anti-NAMPT mAb (“ALT-100”) was tested in a two-hit murine model of chorioamnionitis-induced pulmonary hypertension (PAH). To this end, pups born to chorioamnionitic females were postnatally exposed to LPS and hyperoxia for 14 days, and then housed in room air (RA) for 10-14 days. The LPS/hyperoxia challenged pups were then treated with neutralizing anti-NAMPT mAb or left untreated. Mice kept at room air (RA) served as control. Lung tissue homogenates from the mice were subjected to RT-PCR analysis to assess p-SMAD 1/5/8/9 and STATS expression. The results are shown in FIGS. 8A and 8B, respectively.

As shown in FIG. 8A, lung tissues from LPS/hyperoxia challenged pups (“HO LPS”) showed marked decrease in p-SMAD 1/5/8/9 expression compared to lung tissues from pups that were exposed to control room air (“RA”) or those challenged with LPS at room air (“RA LPS”). In contrast, significant increase in p-SMAD 1/5/8/9 expression was seen in lung tissues obtained from LPS/hyperoxia challenged pups that were treated with anti-NAMPT mAb (“HO LPS NAAMPT”). Thus, the results demonstrate the effectiveness of the anti-NAMPT mAb in restoring p-SMAD expression in a chorioamnionitis-induced PAH model.

As shown in FIG. 8B, lung tissues from LPS/hyperoxia challenged pups (“LPS Hyperoxia”) showed marked increase in STAT3 expression (also seen in human PAH) compared to lung tissues from pups that were exposed to control room air (“RA”) or those challenged with LPS at control room air (“LPS RA”). In contrast, significant decrease in STAT3 expression was seen in lung tissues obtained from LPS-challenged pups that were treated with anti-NAMPT mAb (“LPS NAMPT RA”) and LPS/hyperoxia challenged pups that were treated with anti-NAMPT mAb (“LPS NAMPT Hyperoxia”). Thus, the results demonstrate the effectiveness of the anti-NAMPT mAb in restoring STAT3 expression to baseline in a chorioamnionitis-induced PAH model. Hence, the results described in FIGS. 8A-8B further validate NAMPT as a therapeutic target in chorioamnionitis.

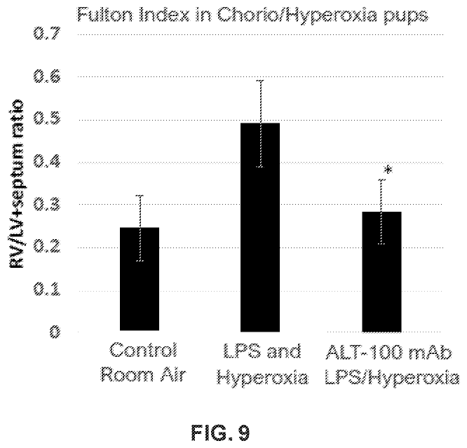

To further validate NAMPT as a therapeutic target in chorioamnionitis, echocardiogram data, hemodynamic data, and fulton index were assessed in pups from the two-hit murine model of chorioamnionitis-induced PAH. Results depicting echo data and hemodynamic data are described in Tables 3-4 and FIG. 9.

TABLE 3
Echo data in murine model of chorioamnionitis-induced PAH

| | | | LPS/ | |
|---|---|---|---|---|
| | | LPS/ | Hyperoxia + | |
| Echo data | Control RA | Hyperoxia | ALT-100 | P value |
| Stroke Volume | 33.93 | 32.57 | 34.11 | NS |
| Cardiac | 15.4 | 15.2 | 14.8 | NS |
| Output/Minute | | | | |
| Ejection Fraction | 59 | 53 | 54 | NS |
| RV Fractional | 0.32 + 0.02 | 0.44 + 0.04 | 0.31 + 0.01 | * 0.04 |
| Wall | | | | |
| Thickening | | | | |
| PAT/PET | 0.34 + 0.01 | 0.25 + 0.03 | 0.34 + 0.01 | * 0.01 |
| TAPSE | 0.669 + 0.03 | 0.51 + 0.03 | 0.67 + 0.03 | * 0.01 |

TABLE 4
Hemodynamic data in murine model
of chorioamnionitis-induced PAH

| Hemodynamic | | LPS/ | LPS/Hyperoxia + | |
|---|---|---|---|---|
| data | Control RA | Hyperoxia | ALT-100 | P value |
| RV/LV ratio | 0.48 | 0.80 | 0.56 | 0.017 |
| RVP | 14.5 | 40.2 | 22.4 | 0.01 |

As shown in Tables 3-4 and FIG. 9, LPS/hyperoxia challenged pups (“LPS Hyperoxia”) showed marked increase in RV fractional wall thickening, RV/LV ratio, and RVP, compared to pups that were exposed to control room air (“Control RA”). In contrast, significant decrease in these parameters was seen in LPS/hyperoxia challenged pups that were treated with anti-NAMPT mAb (“LPS/Hyperoxia+ALT-100”). On the other hand, PAT/PET and TAPSE were decreased in LPS/hyperoxia challenged pups (“LPS Hyperoxia”) compared to pups that were exposed to control room air (“Control RA”), and restoration of these parameters was seen in LPS/hyperoxia challenged pups that were treated with anti-NAMPT mAb (“LPS/Hyperoxia+ALT-100”).

Thus, the results described in Tables 3-4 and FIG. 9 demonstrate the effectiveness of the anti-NAMPT mAb in regulating various parameters of PAH in a chorioamnionitis-induced PAH model, and further validate NAMPT as a therapeutic target in chorioamnionitis.

### Example 12. Assessing NAMPT Expression in Human NAFLD Hepatic Tissues and Murine NASH Tissues

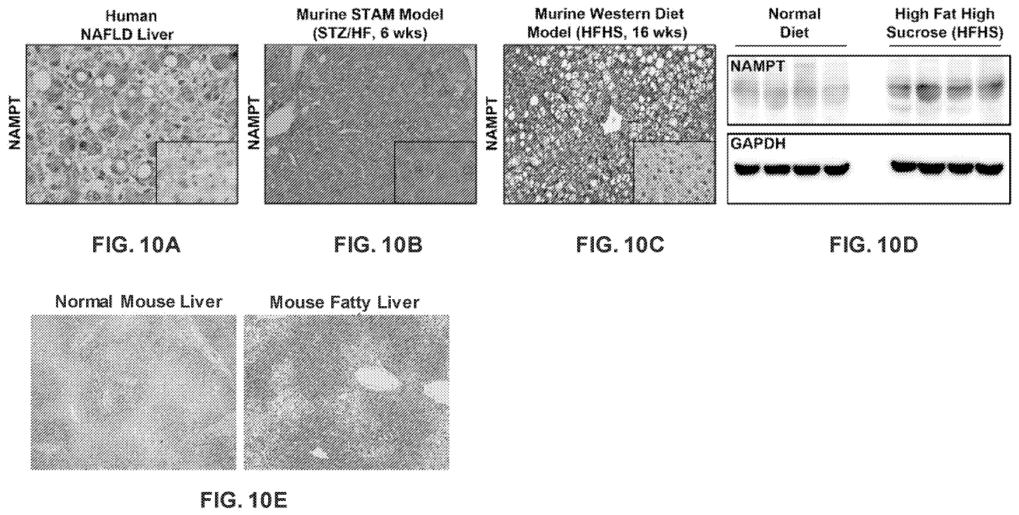

To evaluate the role of NAMPT in nonalcoholic fatty liver disease (NAFLD) and its eventual progression from steatosis to NASH, hepatic NAMPT expression was assessed in humans with NAFLD and in two murine models of NASH. One murine NASH model involved mice that were fed the “western diet” of high fat/high sucrose (HFHS). The second murine model, also called the STAM NASH mouse model, involved mice that were given low dose streptozotocin (STZ) after birth and begun on a high fat diet at week 4 (STZ/HF), which induced NASH by 7 weeks with histological features similar to human NASH (including perisinusoidal fibrosis). Furthermore, hepatic NAMPT expression was assessed in mouse with fatty liver disease. The results are described in FIGS. 10A-10E.

As described in FIG. 10A, IHC analysis of hepatic tissues from five human subjects with NAFLD showed significant increase in NAMPT expression, as compared to normal liver tissues (shown in inset). FIGS. 10B-10D describes NAMPT expression by IHC and western blot analyses in two preclinical murine NASH/fibrosis models. As described in FIG. 10B, NAMPT expression in NASH hepatic tissues is markedly increased in STZ/HF-exposed mice. Similarly, enhanced NAMPT expression was detected by IHC in livers from mice fed a western diet (HFHS, 16 weeks) (FIG. 10C), which was further verified by immunoblot analysis of NAMPT levels in hepatic tissue lysates (FIG. 10D). FIG. 10E describes NAMPT expression by IHC analysis in mouse with fatty liver disease. As described in FIG. 10E, mouse fatty liver showed marked increase in NAMPT expression compared to normal mouse liver. These results are consistent with potential NAMPT involvement in influencing human NASH progression. Thus, the results described in FIGS. 10A-10E demonstrate a dysregulation of NAMPT expression in NAFLD and NASH, indicating a role of NAMPT in NASH pathogenesis.

### Example 13. Antibody-Mediated Neutralization of NAMPT Reduces Hepatic Inflammation, Injury and Fibrosis in a Murine Model of NASH

The results outlined in the foregoing example demonstrates a role of NAMPT in NASH pathogenesis, thus indicating the potentials of NAMPT as a therapeutic target in NASH. Next, to explore the potentials of NAMPT as a therapeutic target in NASH, the efficacy of neutralizing circulating anti-NAMPT mAb was assessed in STZ/HF (STAM) mice. STZ/HF mice were C57/B6 mice that were challenged with streptozotocin (STZ) 2 days after birth, and maintained on a high fat diet (HF). Between weeks 9-12, the STZ/HF mice were intraperitoneally (i.p.) injected weekly with 0.4 mg/kg of a humanized anti-NAMPT mAb (ALT-100) or vehicle control (control IgG1 Ab). This strategy was designed to target development of NASH/hepatic fibrosis and then compare the findings to daily orally-delivered Telmisartan, an angiotensin receptor blocker known to reduce preclinical NASH, which served as a positive control.

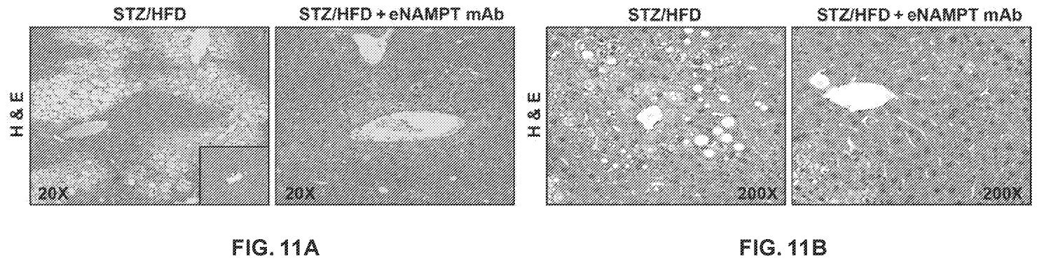

First, liver tissues obtained from STZ/HF-exposed mice (“STZ/HF”) and anti-NAMPT mAb-treated STZ/HF mice (“STZ/HF+eNAMPT mAb”) were subjected to H&E staining to assess NASH-mediated hepatic injury. Results from the analyses are described in FIGS. 11A-11B. As described in FIGS. 11A-11B, compared to hepatic tissues from control mice (FIG. 11A, left panel, inset), hepatic tissues from STZ/HF mice (FIGS. 11A-11B, left panels) showed significant hepatic steatosis, intrahepatic fat globules and hepatocyte injury/ballooning. However, hepatic tissues from anti-NAMPT mAb-treated STZ/HF mice (FIGS. 11A-11B, right panels) showed marked reduction in hepatic steatosis, intrahepatic fat globules and hepatocyte injury/ballooning (comparable to Telmisartan protection, data not shown). Thus, the results described in FIGS. 11A-11B show that neutralization of NAMPT by anti-NAMPT mAb is effective in attenuating histologic progression of steatosis and NASH in STZ/HF mice.

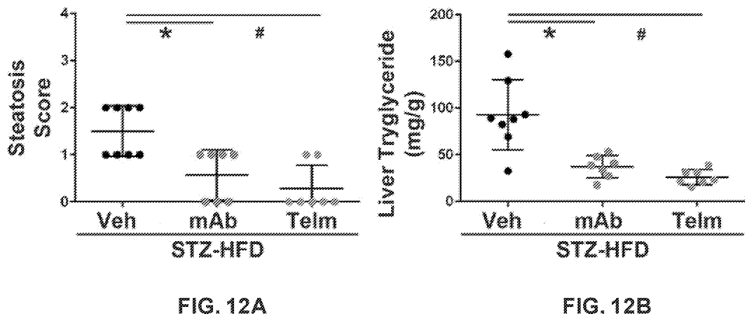

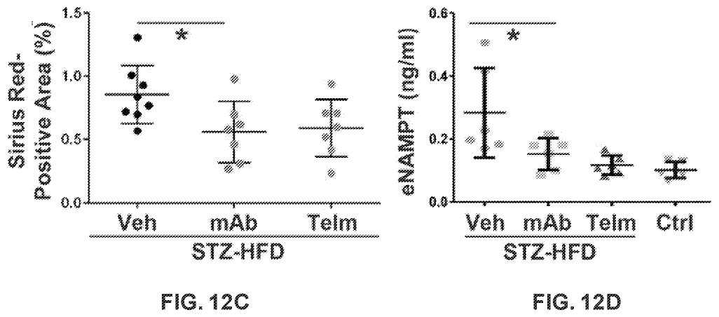

Next, liver tissues obtained from anti-NAMPT mAb-treated STZ/HF mice (“mAb”), vehicle injected STZ/HF mice (“Veh”) or Telmisartan-treated STZ/HF mice (“Telm”) were analyzed for steatosis score and liver triglyceride level. Results from the analyses are depicted in FIGS. 12A-12B. As described in FIG. 12A, anti-NAMPT mAb treated mice showed a significant reduction in steatosis score, compared to mice that were injected with vehicle control (p<0.01). Also, as described in FIG. 12B, a robust decrease in liver triglyceride level was observed in anti-NAMPT mAb-treated mice compared to those injected with vehicle control (p<0.01). Thus, the results described in FIGS. 12A-12B show that neutralization of NAMPT by anti-NAMPT mAb effectively reduces inflammatory indices in NASH.

Next, liver tissues obtained from anti-NAMPT mAb-treated STZ/HF mice (“mAb”), vehicle injected STZ/HF mice (“Veh”) or Telmisartan-treated STZ/HF mice (“Telm”) were subjected to sirius red staining for collagen, a fibrosis marker, to assess NASH-mediated hepatic fibrosis. Results from the analyses are shown in FIG. 12C. As shown in FIG. 12C, a significant reduction in percentage of sirius red-positive area, which indicates a significant reduction in expression of the fibrosis marker collagen, was observed in liver tissues from anti-NAMPT mAb-treated mice compared to mice that were injected with vehicle control (p<0.05). Thus, the results described in FIG. 12C shows that neutralization of NAMPT by anti-NAMPT mAb is effective in attenuating NASH-mediated hepatic fibrosis.

Next, plasma levels of NAMPT was assessed in anti-NAMPT mAb treated STZ/HF mice (“mAb”), vehicle-injected STZ/HF mice (“Veh”), Telmisartan-treated STZ/HF mice (“Telm”) or control mice that were not exposed to STZ/HF (“Ctrl”). The results are described in FIG. 12D. As described in FIG. 12D, compared to untreated control mice, vehicle injected STZ/HF mice showed a marked increase in NAMPT plasma level, which was significantly decreased by treatment with anti-NAMPT mAb. Thus, the results demonstrate a dysregulation of plasma NAMPT level in NASH, and also validate a role of NAMPT in NASH pathogenesis

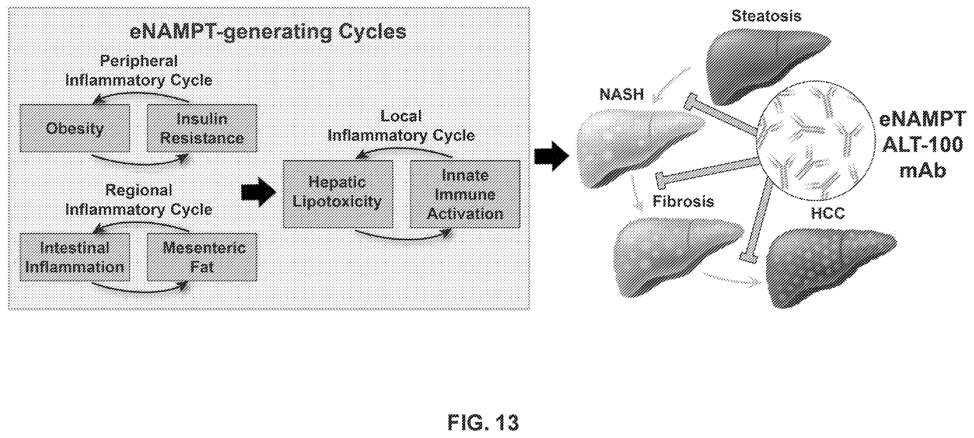

Overall, the results of the aforementioned study, as outlined in FIGS. 10A-10E, 11A-11B and 12A-12D, demonstrate NAMPT as a novel NASH therapeutic target that regulates inflammation and fibrosis, and also shows the effectiveness of the anti-NAMPT antibody in attenuating NASH-mediated hepatic inflammation, injury and fibrosis, thus validating NAMPT as a therapeutic target in NASH. This is further summarized in the schematics provided in FIG. 13. As described in FIG. 13, the three cycles of inflammation that result in NASH include: peripheral inflammatory cycle caused by obesity and insulin resistance; regional inflammatory cycle caused by intestinal inflammation and activation of mesenteric adipocytes; and local inflammatory cycle, which is intrahepatic and caused by hepatic lipotoxicity and innate immune cell activation. Intestinal inflammation is caused by the “leaky gut syndrome” and luminal contents draining into the liver via the portal vein. NAMPT may be involved in each inflammatory cycle, robustly produced by adipocytes, intestinal epithelium, and by activated hepatocytes. As a DAMP, NAMPT triggers TLR4/NFkB inflammatory signaling to locally produce chemokines/cytokines and growth factors that promote hepatocyte injury, fibrosis and transition to neoplasia, processes that are significantly attenuated by the NAMPT-neutralizing mAb.

The compelling preclinical and clinical data presented in the present disclosure strongly implicate NAMPT as an attractive target to reduce risk of NAFLD progression to NASH, as: (i) NAMPT plasma levels and hepatic NAMPT expression are significantly elevated in murine NASH models; (ii) humans with NASH and NASH cirrhosis exhibit increased hepatic NAMPT expression; (iii) a NAMPT-neutralizing mAb (ALT-100), currently in the process of GMP manufacturing, reduced lung and renal fibrosis (see, Example 14 below) and multiple indices of NAFLD progression to NASH fibrosis. Thus, the humanized, sequence-optimized, NAMPT-neutralizing mAb (e.g., ALT-100 or Ab3) may represent a safe and effective approach for slowing or halting progression to life threatening NASH and HCC.

The present disclosure provides a platform (ENAMPTOR™ platform), which in addition to a NAMPT inhibitor, such as a humanized anti-NAMPT-neutralizing antibody (e.g., the ALT-100 mAb), comprises a NAMPT plasma biomarker assay and a NAMPT genotyping assay. It has been shown that both plasma NAMPT levels (Bime et al., Am J Respir Crit Care Med (2018), 197:1421-1432) and five NAMPT promoter SNPs predict acute respiratory distress syndrome (ARDS) (Bajwa et al., Crit Care Med (2007), 35:1290-1295) and pulmonary arterial hypertension mortality and are risk factors for severe inflammatory injury. Each NAMPT SNP alters NAMPT promoter activity in response to mechanical stress and hypoxia (via HIF-2α) (Sun et al., Am J Respir Cell Mol Biol (2014), 51:660-667; Sun et al., Am J Respir Cell Mol Biol (2020), 63:92-103). Each SNP has a minor allelic frequency >1% in Blacks, with three NAMPT SNPs significantly over-represented in Blacks. The precision medicine platform disclosed herein allows identification of patients most likely to respond to the NAMPT therapy (e.g., treatment with ALT-100 mAb). Both NAMPT plasma levels and NAMPT risk genotypes may represent novel risk factors in progression/mortality of inflammatory conditions, such as the progression of NAFLD to NASH. The precision medicine platform disclosed herein may, for example, be of significant value in stratifying subjects (e.g., NASH subjects) for enrollment into a clinical trial of the treatment with an NAMPT inhibitor (e.g., NAMPT-neutralizing ALT-100 mAb).

### Example 14. Antibody-Mediated Neutralization of NAMPT Reduces Ureteral Obstruction-Induced Murine Renal Fibrosis

Next, the efficacy of neutralizing circulating anti-NAMPT mAb in reducing or preventing the severity of organ fibrosis was assessed in a ureteral obstruction-induced murine renal fibrosis model. Unilateral ureteral obstruction (UUO) is a well-characterized disease model for renal fibrosis with key pathophysiological features of chronic kidney disease, tubular necrosis and inflammatory cell infiltration within a relatively short period. NAMPT expression and efficacy of NAMPT-neutralizing ALT-100 mAb on reducing kidney inflammation and fibrosis was assessed in the preclinical murine UUO model.

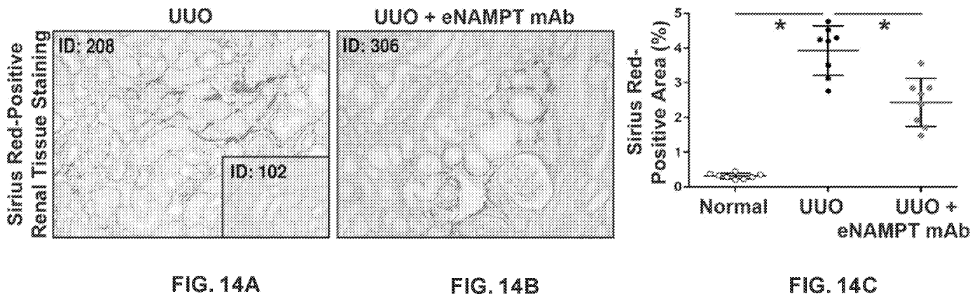

To this end, three groups of 8 female C57BL/6 mice (seven-week-old) were surgically challenged with implementation of ureteral obstruction to initiate renal fibrosis. Wild-type (WT) mice received 0.4 mg/kg of ALT-100 mAb (i.p.) at day 1 and 8 post-surgery, with all mice sacrificed at day 14. UUO-mediated increase in Sirius red-positive renal tissue staining was assessed as an indicator of renal fibrosis. The results are described in FIGS. 14A-14C. As described in FIGS. 14A-14C, the NAMPT mAb ALT-100 significantly reduced Sirius red-positive staining in the UUO model, with an accompanying reduction in kidney hydroxyproline content although this did not reach statistical significance (data not shown).

Thus, the results of the aforementioned study underscore the effectiveness of neutralizing circulating anti-NAMPT mAb in reducing the severity of organ fibrosis (e.g., renal fibrosis).

### Example 15. Further Research Design and Methods

Initial explorations of NAMPT involvement in NAFLD (as outlined in the foregoing Examples) revealed marked increases in hepatic NAMPT expression both in humans with NAFLD as well as in two murine models of NASH. One murine NASH model involved mice fed the “western diet” of high fat/high sucrose (HFHS). The second murine model involved mice given low dose streptozotocin (STZ) after birth and begun on a high fat diet at week 4 (STZ/HF), also called the STAM NASH mouse model. Importantly, the ALT-100 mAb reduced multiple indices of NASH severity in the murine STAM model. As NAMPT is a novel upstream therapeutic target in multiple inflammatory disorders, the NAMPT-neutralizing humanized ALT-100 mAb should retard NAFLD progression to NASH and to fibrosis/cirrhosis. This Phase I study is designed to confirm the NAMPT-neutralizing ALT-100 mAb as a therapeutic strategy to retard NAFLD progression to NASH and hepatic fibrosis/cirrhosis. The therapeutic delivery of ALT-100 mAb in preclinical NASH models will also be optimized.

Study (1): Optimizing NAMPT mAb Delivery (ALT-100) in a Preclinical Murine STAM Model of NASH (STZ/HFD).

The NAMPT-neutralizing mAb will be assessed at three doses and two time points (weeks 5 or 9) in the preclinical murine STAM model of NASH. Readouts for mAb efficacy will be significant attenuation of liver inflammation, hepatocyte injury, including known indicators of steatosis and fibrosis, and mortality.

Study (2): Optimizing Anti-NAMPT mAb Delivery (ALT-100) in a Preclinical Rat AMLN Model of NASH (HFHCD).

The dosing/timing of the anti-NAMPT mAb will be optimized in a preclinical rat western diet NAFLD model (high fat, high fructose, high cholesterol diet or HFHCD). Readouts for mAb efficacy will be significant reductions in liver inflammation, hepatocyte injury including known indicators of steatosis and fibrosis.

Phase II Planning.

Successful completion of Phase I studies will be followed by Phase II, where pharmacokinetic (PK), pharmacodynamic (PD), and safety (toxicology) profiles of ALT-100 mAb will be assessed, and a CMC infrastructure to support clinical development will be established. Successful completion of the Phase II studies will be followed by validation of ALT-100 mAb as a therapeutic approach in healthy volunteers (Phase I trial) and in subjects with NASH (Phase II trial).

Biologic Rationale.

Therapeutic advances in NAFLD progression and management now highlight the role of inflammatory pathway activation (Schuster et al., “Triggering and resolution of inflammation in NASH,” Nat Rev Gastroenterol Hepatol, 15:349-364 (2018)) in NASH development, hepatic fibrogenesis and progression to HCC. Utilizing systems biology and genomic strategies, the present inventors identified extracellular nicotinamide phosphoribosyltransferase (eNAMPT) as a novel damage-associated molecular pattern protein (DAMP) that potently regulates innate immunity-mediated inflammation and fibrosis via ligation of TLR4 (Ye et al., “Pre-B-cell colony-enhancing factor as a potential novel biomarker in acute lung injury,” Am J Respir Crit Care Med, 171:361-370 (2005); Camp et al., “Unique Toll-Like Receptor 4 Activation by NAMPT/PBEF Induces NFkappaB Signaling and Inflammatory Lung Injury,” Sci Rep, 5:13135 (2015); Oita et al., “Novel Mechanism for Nicotinamide Phosphoribosyltransferase Inhibition of TNF-alpha-mediated Apoptosis in Human Lung Endothelial Cells,” Am J Respir Cell Mol Biol, 59:36-44 (2018)). NAMPT plays an essential role in multi-organ inflammation, injury and fibrosis (lung, liver, renal, cardiac) and in cancer (Sun et al., “Role of secreted extracellular nicotinamide phosphoribosyltransferase (eNAMPT) in prostate cancer progression: Novel biomarker and therapeutic target,” EBioMedicine, 61:103059 (2020)) (FIG. 1). The same NAMPT protein, when intracellular as iNAMPT, enzymatically regulates NAD metabolism, and has been targeted with SMI in clinical cancer trials, but failed due to lack of benefit and toxicity (Sun et al., “Role of secreted extracellular nicotinamide phosphoribosyltransferase (eNAMPT) in prostate cancer progression: Novel biomarker and therapeutic target,” EBioMedicine, 61:103059 (2020)). The studies described herein focus on NAMPT (e.g., eNAMPT) as a key regulator of NAFLD progression to NASH and attractive therapeutic target to reduce NAFLD progression to later stages. Preclinical studies indicate increased hepatic NAMPT expression in murine NAFLD/NASH models and in humans with NAFLD. Furthermore, a NAMPT-neutralizing mAb (ALT-100 mAb) reduced multiple indices of NASH severity, suggesting ALT-100 mAb may be a viable and novel approach to address the urgent unmet need to prevent NASH progression/lethality.

Proposed Product.

Although the therapeutic pipeline for NASH is expanding, successful targeting of progressive NAFLD remains an unmet need. NAMPT generated by mesenteric adipose tissues and a “leaky gut” should directly activate the TLR4/NFkB-dependent inflammatory cascade (Camp et al., “Unique Toll-Like Receptor 4 Activation by NAMPT/PBEF Induces NFkappaB Signaling and Inflammatory Lung Injury,” Sci Rep, 5:13135 (2015); Oita et al., “Novel Mechanism for Nicotinamide Phosphoribosyltransferase Inhibition of TNF-alpha-mediated Apoptosis in Human Lung Endothelial Cells,” Am J Respir Cell Mol Biol, 59:36-44 (2018)), thereby contributing to hepatic transition from steatosis to NASH and hepatic fibrosis (FIG. 13). NAMPT is a highly druggable therapeutic target and the present inventors have developed the ALT-100 mAb, a humanized NAMPT-neutralizing mAb, as a novel therapy for NASH treatment. ALT-100 mAb effectively reduces TLR4/NFkB inflammatory cascade activation in preclinical ARDS/VILI (Hong et al., “Essential role of pre-B-cell colony enhancing factor in ventilator-induced lung injury,” Am J Respir Crit Care Med, 178:605-617 (2008); Quijada et al., “Endothelial eNAMPT Amplifies Preclinical Acute Lung Injury: Efficacy of an eNAMPT-Neutralising mAb,” Eur Respir J, 2002536 (2020)), pulmonary hypertension (Chen et al., “Nicotinamide Phosphoribosyltransferase Promotes Pulmonary Vascular Remodeling and Is a Therapeutic Target in Pulmonary Arterial Hypertension,” Circulation, 135:1532-1546 (2017); Sun et al., “Direct Extracellular NAMPT Involvement in Pulmonary Hypertension and Vascular Remodeling. Transcriptional Regulation by SOX and HIF-2alpha,” Am J Respir Cell Mol Biol, 63:92-103 (2020)), radiation-induced lung injury and prostate cancer (Sun et al., “Role of secreted extracellular nicotinamide phosphoribosyltransferase (eNAMPT) in prostate cancer progression: Novel biomarker and therapeutic target,” EBioMedicine, 61:103059 (2020)). The anti-NAMPT ALT-100 mAb was selected after comprehensive in vitro and in vivo screening of 50 NAMPT-neutralizing mAbs derived from 2 parental murine mAbs (Fusion Antibodies Inc, Belfast, UK). The anti-NAMPT ALT-100 mAb has undergone sequence optimization to remove immunogenic sequences and exhibits strong NAMPT binding potency with an SPR-derived Kd of 4.5 nM. Supported by strong preclinical/clinical data and utilizing 2 preclinical NASH models, studies proposed herein are designed provide further proof of concept for anti-NAMPT ALT-100 mAb as a therapeutic strategy to reduce NAFLD progression from steatosis to NASH to hepatic fibrosis/cirrhosis.

Innovation.

First, targeting extracellular NAMPT (e.g., eNAMPT) as a promising approach to prevent NAFLD and progression from steatosis to NASH and hepatic fibrosis is extraordinarily innovative and, to our knowledge, there are no NASH therapeutic competitors utilizing this biologic approach. Second, our hypothesis, illustrated in FIG. 13, is very innovative and highlights the role of eNAMPT as a DAMP responding to increased leaky gut syndrome, activated mesenteric adipose tissues, steatosis-related lipotoxicity, ROS, and hypoxia. Via TLR4 ligation and NFkB inflammatory signaling, NAMPT contributes to induction of a NASH-progressing microenvironment and functionally impacts NAFLD progression. NAMPT neutralization has the extremely advantageous property of dampening the inflammatory burden produced by peripheral, intestinal (leaky gut-derived endotoxins), and hepatic-localized inflammatory mechanisms. Third, the anti-NAMPT mAb itself is innovative and has been sequence-optimized to reduce immunogenicity, with our pharmacodynamic/pharmacokinetic studies in Sprague Dawley rats revealing a mAb half-life of 14-18 days without any observed toxicity, even when up to a 50 mg/kg doses. Finally, the requirement for a precision medicine approach has been recognized as a key element to successfully target NAFLD. The present inventors have developed the ENAMPTOR™ precision medicine platform consisting of: i) an NAMPT (e.g., eNAMPT) plasma biomarker assay; and ii) a 5 SNP NAMPT genotyping assay both which we have shown as risk factors for severe inflammatory injury. We speculate that the ENAMPTOR™ precision medicine platform, comprised of the eNAMPT plasma biomarker/genotyping assays and the humanized anti-NAMPT ALT-100 mAb, will allow identification of NAFLD patients most likely to respond to anti-NAMPT ALT-100 mAb, an additional source of innovation.

### Research Design:

The results presented in the foregoing Examples demonstrate NAMPT as a highly druggable target and underscore the effectiveness of a humanized NAMPT-neutralizing ALT-100 mAb in limiting the lethal transition from steatosis to NASH and NASH cirrhosis. The ALT-100 mAb has dramatic efficacy in preclinical inflammatory models of disease, such as ARDS, radiation-induced lung injury, pulmonary hypertension, and prostate cancer (Quijada et al., “Endothelial eNAMPT Amplifies Preclinical Acute Lung Injury: Efficacy of an eNAMPT-Neutralising mAb,” Eur Respir J, 2002536 (2020); Sun et al., “Role of secreted extracellular nicotinamide phosphoribosyltransferase (eNAMPT) in prostate cancer progression: Novel biomarker and therapeutic target,” EBioMedicine, 61:103059 (2020)). Next, anti-NAMPT mAb will be tested in preclinical mouse models of NASH by: (1) evaluating ALT-100 efficacy and optimal dosing/timing in the murine STAM model of NASH (STZ/HFD) (see, FIGS. 10A-10E, 11A-11B, and 12A-12D); and (2) evaluating ALT-100 efficacy and optimal dosing/timing in a rat AMLN model (HFHCD) that mimics the western diet and induces the full spectrum of NAFLD (steatosis, NASH, hepatic fibrosis). Common endpoints include significant reductions in liver dysfunction/injury, inflammation, and fibrosis. ALT-100 mAb will be given weekly as pharmacokinetic studies in rats demonstrated that ALT-100 mAb half-life is 16-18 days.

### Study (1)—Optimizing NAMPT-Neutralizing mAb Dosing in Murine STAM Model (STZ/HF) of NASH

Study (1) Rationale:

NAMPT will be tested as a highly druggable target in NASH utilizing a preclinical murine model of NASH (STAM). Specifically, mice are injected 2 days after birth with a low dose of STZ to induce b-cell injury followed by high diet feeding at 4 weeks of age. This model develops a full spectrum of NAFLD progression with simple steatosis seen at 5 weeks, NASH at 7 weeks, fibrosis at 9 weeks post STZ. Although not shown in this disclosure, HCC develops by 16 weeks post STZ. Preliminary data (FIGS. 10A-10E, and 11A-11B) depict the development of NASH in this murine STAM model and the preliminary success of anti-NAMPT mAb in reducing NASH. For Study (1) (i.e., for evaluating ALT-100 efficacy and optimal dosing/timing in murine STAM model of NASH), weekly intraperitoneal injections of the anti-NAMPT mAb will be assessed for efficacy in providing significant reduction in liver damage and NAFLD progression to NASH. The three anti-NAMPT mAb doses for these studies were chosen based upon a number of preclinical studies for the indications of pulmonary hypertension and radiation fibrosis and will lead us to determine optimal dosing of ALT-100 mAb in effectively reducing NASH progression, enabling STTR Phase II studies and Phase 1B Clinical Trials in NASH patients. In addition, this study will address two temporal NASH stages in this STAM model and initiate anti-NAMPT-neutralizing mAb delivery at week 5 as NASH-targeting strategy and in a separate group at week 8 as a fibrosis-targeting strategy.

Study (1) Experimental Design and Methods:

8 groups of C57BL/6 mice (20 mice in each group) will be exposed to 200 μg of STZ 2 days after birth via single subcutaneous injection, followed by HF feeding at 4 weeks of age. Group I will receive weekly IP-delivered control IgG₁ Ab 1.0 mg/kg beginning at week 5. Group VIII will receive weekly IP-delivered control IgG₁ Ab 1.0 mg/kg beginning at week 8. Groups II and V will receive the ALT-100 mAb at 0.4 mg/kg beginning at week 5 (Group II) or week 8 (Group V). Groups III and VI will receive ALT-100 mAb at 1.0 mg/kg at week 5 (Group III) or week 8 (Group VI). Finally, Groups IV and VII will receive the ALT-100 mAb at 4.0 mg/kg at week (Group IV) or week 8 (Group VII). All animals will be sacrificed at 20 weeks. Clinically equivalent endpoints and readouts of mAb efficacy will be reductions in: i) liver H&E histological analysis for NAFLD Activity Score (NAS); ii) liver to body weight ratios; iii) plasma ALT, AST and cholesterol; iv) liver triglyceride and cholesterol; v) Sirius staining for % area of collagen staining (fibrosis score); and vi) mortality (expected to be 30% at 16 weeks and 45% at 20 weeks in STZ/HF, IgG₁ group). Based upon preliminary studies, these experiments should detect a reduction of 40% in fibrosis indices with a 90% confidence at an alpha of 0.1 given we are utilizing 20 mice per intervention.

Study (1) Anticipated Results, Pitfalls and Alternatives:

STZ-challenged mice with HF feeding should develop NASH and untreated mice will begin to exhibit NASH progression and mortality within 20 weeks. Compared to the IgG₁ control, ALT-100 mAb should significantly and dose-dependently reduce NASH progression with potential reversal which is dependent upon the timing of anti-NAMPT mAb initiation. Milestones: M1) In the STZ/HF murine model, the anti-NAMPT-neutralizing ALT-100 mAb will demonstrate a dose-dependent reduction in NAFLD severity/progression. M2) Optimal anti-NAMPT ALT-100 mAb dose will be established. M3) The importance of early eNAMPT ALT-100 mAb intervention will be established.

### Study (2)—Optimizing Anti-NAMPT mAb Dosing in the Rat AMLN Model (HFHCD) of NASH

### Study (2) Rationale:

Complementing the anti-NAMPT mAb studies in the murine STAM model (STZ/HF) described in Study (1) that incorporates both diabetes and obesity (the two major risk factors for NAFLD), Study (2) utilizes a dietary-induced rat model, the AMLN diet which consists of 40% kcal fat (primarily Primex hydrogenated vegetable oil shortening, i.e. trans-fat), 20% kcal fructose and 2% w/w cholesterol (HFHCD), one of the best diet-induced NASH models for preclinical investigations. The HFHCD animals exhibit metabolic features that are seen in human NAFLD covering the full spectrum of NAFLD over 16-20 weeks (steatosis, NASH, and hepatic fibrosis) (Clapper et al., “Diet-induced mouse model of fatty liver disease and nonalcoholic steatohepatitis reflecting clinical disease progression and methods of assessment,” Am J Physiol Gastrointest Liver Physiol, 305:G483-495 (2013); Kristiansen et al., “Obese diet-induced mouse models of nonalcoholic steatohepatitis-tracking disease by liver biopsy,” World J Hepatol, 8:673-684 (2016); Trevaskis et al., “Glucagon-like peptide-1 receptor agonism improves metabolic, biochemical, and histopathological indices of nonalcoholic steatohepatitis in mice,” Am J Physiol Gastrointest Liver Physiol, 302:G762-772 (2012); Zeng et al., “Effects and therapeutic mechanism of Yinzhihuang on steatohepatitis in rats induced by a high-fat, high-cholesterol diet. J Dig Dis, 21:179-188 (2020)). Although different stages of NAFLD are developed much slower in the AMLN/HFHCD model (Study (2)), compared to STAM (STZ/HF) model in Study (1), it is one of the newest models that faithfully mimics the human disease without toxic chemical injection. This AMLN model (HFHCD) is also superior to the HFHS model (FIGS. 10C-10D) and exhibits greater lobular inflammation and more severe NASH. As with Study (1), Study (2) will assess the efficacy of weekly intraperitoneal injections of the anti-NAMPT mAb in providing significant reduction in liver damage. These studies will allow determination of optimal dosing of ALT-100 mAb in effectively reducing NASH progression, enabling STTR Phase II studies and Phase 1B Clinical Trials in NASH patients.

Study (2) Experimental Design and Methods:

A total of 8 groups (Group I-VIII) of Sprague-Dawley rats (20 rats in each group) will be fed a diet containing 40% fat (˜18% trans-fat), 22% fructose and 2% cholesterol (Research Diets, Inc, Cat #D09100301). Two groups (Group IX-X) of control rats will be maintained on a normal fat control diet from the same company (cat #D09100304). Beginning at week 12, Group I-IV will receive weekly IP-delivery of either control IgG₁ Ab (1.0 mg/kg) (Group I), the ALT-100 mAb (0.4 mg/kg) (Group II), the ALT-100 mAb (1.0 mg/kg) (Group III), or the ALT-100 mAb (4.0 mg/kg) (group IV). Beginning at week 16, Group V-VIII will receive weekly IP-delivered of either control IgG₁ Ab (1.0 mg/kg) (group V), the ALT-100 mAb (0.4 mg/kg) (group VI), the ALT-100 mAb (1.0 mg/kg) (group VII), or the ALT-100 mAb (4.0 mg/kg) (Group VIII). Control groups receiving normal chow (Group IX-X) will receive weekly IP-delivery of either control IgG₁ Ab (1.0 mg/kg) (Group IX) or the ALT-100 mAb (1.0 mg/kg) (Group X) beginning at week 12. All animals will be sacrificed at 30 weeks. Clinically equivalent endpoints and readouts of mAb efficacy will be reductions in: i) liver H&E histological analysis for NAFLD Activity Score (NAS); ii) liver to body weight ratios; iii) plasma ALT, AST and cholesterol; iv) liver triglyceride and cholesterol; v) Sirius staining for % area of collagen staining (fibrosis score). Based upon our preliminary studies, these experiments should detect a reduction of 40% in fibrosis indices with a 90% confidence at an alpha of 0.1 given we are utilizing 20 rats per intervention.

Study (2) Anticipated Results, Potential Pitfalls and Alternatives:

The development of NASH should be dose-dependently reduced with ALT-100 mAb treatment, compared to the IgG₁ controls. Milestones: M4) In the AMLN (HFHCD) rat model, the anti-NAMPT-neutralizing ALT-100 mAb will demonstrate dose-dependent reduction in NASH severity/progression. M5) The optimal NASH-reducing ALT-100 mAb dose in the AMLN (HFHCD) rat model will be determined. M6) The importance of early eNAMPT ALT-100 mAb intervention will be established in the rat AMLN (HFHCD) model.

### Statistical Considerations

The sample size justification of 20 mice/group is based on:

(i) an anticipated take rate of at least 80% (n=16 NASH/group). The presence of NASH will be analyzed by fitting a mixed linear model of fibrosis volume vs. time for each mouse. The resulting slopes (fibrosis rate) will be compared using a factorial model.

(ii) comparisons between “no treatment” and “treatment” responses. 80% statistical power will allow detection of a standardized decrease of 1.325 between the means of two groups (difference divided by SD) assuming a two-sided alpha of 0.05. Statistical tests will be performed using Bonferroni Multiple Comparison Test. P values <0.05 will be considered statistically significant.

1. A method of identifying a subject having or at risk of developing cardiac ischemia, traumatic brain injury, cancer, chorioamnionitis, nonalcoholic steatohepatitis (NASH), or renal fibrosis, the method comprising:
a) obtaining a sample from a subject at risk of having or developing cardiac ischemia, traumatic brain injury, cancer, chorioamnionitis, NASH, or renal fibrosis; and
b) detecting the presence of at least one single nucleotide polymorphism (SNP) associated with human nicotinamide phosphoribosyl transferase (NAMPT) in the sample, wherein the SNP is selected from the group consisting of rs7789066, rs61330082, rs9770242, rs59744560, rs116647506, rs1319501, rs114382471, and rs190893183.

2. The method of claim 1, wherein the subject has cardiac ischemia, traumatic brain injury, cancer, chorioamnionitis, NASH, or renal fibrosis.

3. The method of claim 1 or 2, wherein the subject has at least 2 SNPs, at least 3 SNPs, at least 4 SNPs, at least 5 SNPs, at least 6 SNPs, at least 7 SNPS, or at 8 SNPs selected from the group consisting of rs7789066, rs61330082, rs9770242, rs59744560, rs116647506, rs1319501, rs114382471, and rs190893183.

4. The method of any one of claims 1-3, comprising detecting at least 2 SNPs selected from the group consisting of rs7789066, rs61330082, rs9770242, rs59744560, rs116647506, rs1319501, rs114382471, and rs190893183.

5. The method of any one of claims 1-4, comprising detecting at least one SNP selected from the group consisting of rs7789066, rs61330082, rs9770242, and rs59744560.

6. The method of any one of claims 1-5, comprising detecting at least one SNP selected from the group consisting of rs116647506, rs1319501, rs114382471, and rs190893183.

7. The method of any one of claims 1-6, wherein the subject is of African descent.

8. The method of any one of claims 1-7, wherein the detecting comprises using polymerase chain reaction (PCR), an SNP microarray, SNP-restriction fragment length polymorphism (SNP-RFLP), dynamic allele-specific hybridization (DASH), primer extension (MALDI-TOF) mass spectrometry, single strand conformation polymorphism, and/or new generation sequencing (NGS).

9. The method of any one of claims 1-7, wherein the detecting comprises contacting the sample with an oligonucleotide probe that selectively hybridizes to a nucleotide sequence comprising the SNP, or a nucleotide sequence complementary thereto, and detecting selective hybridization of the oligonucleotide probe.

10. The method of claim 9, wherein the oligonucleotide probe comprises a detectable label, and wherein detecting selective hybridization of the probe comprises detecting the detectable label.

11. The method of claim 10, wherein the detectable label comprises a fluorescent label, a luminescent label, a radionuclide, or a chemiluminescent label.

12. The method of claim 9, wherein the oligonucleotide probe comprises a bilabeled oligonucleotide probe, comprising a fluorescent moiety and a fluorescent quencher.

13. The method of any one of claims 1-12, wherein the SNP is associated with a NAMPT promoter activity level that is higher than a baseline NAMPT promoter activity level, optionally wherein the baseline NAMPT promoter activity level is a level associated with a subject that does not have cardiac ischemia, traumatic brain injury, cancer, chorioamnionitis, NASH, or renal fibrosis.

14. The method of any one of claims 1-13, wherein the sample is a plasma sample.

15. A method of treating a subject having or at risk of having cardiac ischemia, traumatic brain injury, cancer, chorioamnionitis, NASH, or renal fibrosis, the method comprising:
a) obtaining a sample from a subject having or at risk of having cardiac ischemia, traumatic brain injury, cancer, chorioamnionitis, NASH, or renal fibrosis;
b) detecting the presence or absence of at least one SNP in the sample, wherein the SNP is selected from the group consisting of rs7789066, rs61330082, rs9770242, rs59744560, rs116647506, rs1319501, rs114382471, and rs190893183, and wherein the presence of the at least one SNP indicates that the subject has or is at risk for developing cardiac ischemia, traumatic brain injury, cancer, chorioamnionitis, NASH, or renal fibrosis; and
c) administering an effective amount of a NAMPT inhibitor to the subject having or at risk for developing cardiac ischemia, traumatic brain injury, cancer, chorioamnionitis, NASH, or renal fibrosis.

16. The method of claim 15, wherein the sample is a plasma sample.

17. The method of claim 15 or 16, comprising detecting at least 2 SNPs, at least 3 SNPs, at least 4 SNPs, at least 5 SNPs, at least 6 SNPs, at least 7 SNPS, or at least 8 SNPs selected from the group consisting of rs7789066, rs61330082, rs9770242, rs59744560, rs116647506, rs1319501, rs114382471, and rs190893183.

18. The method of any one of claims 15-17, wherein the detecting the presence or absence of at the least one SNP comprises detecting the presence or absence of at least one SNP selected from the group consisting of rs7789066, rs61330082, rs9770242 and rs59744560.

19. The method of any one of claims 15-18, wherein the detecting the presence or absence of at the least one SNP comprises detecting the presence or absence of at least one SNP selected from the group consisting of rs116647506, rs1319501, rs114382471, and rs190893183.

20. The method of any one of claims 15-19, wherein the subject is of African descent.

21. The method of any one of claims 15-20, wherein the detecting comprises using polymerase chain reaction (PCR), a SNP microarray, SNP-restriction fragment length polymorphism (SNP-RFLP), dynamic allele-specific hybridization (DASH), primer extension (MALDI-TOF) mass spectrometry, single strand conformation polymorphism, or new generation sequencing (NGS).

22. The method of any one of claims 15-21, wherein the detecting comprises contacting the sample with an oligonucleotide probe that selectively hybridizes to a nucleotide sequence comprising the SNP, or a nucleotide sequence complementary thereto, and detecting selective hybridization of the oligonucleotide probe.

23. The method of claim 22, wherein the oligonucleotide probe comprises a detectable label, and wherein detecting selective hybridization of the probe comprises detecting the detectable label.

24. The method of claim 23, wherein the detectable label comprises a fluorescent label, a luminescent label, a radionuclide, or a chemiluminescent label.

25. The method of claim 22, wherein the oligonucleotide probe comprises a bilabeled oligonucleotide probe, comprising a fluorescent moiety and a fluorescent quencher.

26. The method of any one of claims 15-25, wherein the SNP is associated with a NAMPT promoter activity level that is higher than a baseline NAMPT promoter activity level.

27. The method of claim 26, wherein the baseline NAMPT promoter activity level is a level associated with a subject that does not have cardiac ischemia, traumatic brain injury, cancer, chorioamnionitis, NASH, or renal fibrosis.

28. The method of any one of claims 15-27, wherein the NAMPT inhibitor is an anti-NAMPT antibody.

29. The method of claim 28, wherein the anti-NAMPT antibody comprises:
CDR1, CDR2, and CDR3 domains of a heavy chain variable region having an amino acid sequence set forth as SEQ ID NO: 2; and
CDR1, CDR2, and a CDR3 domains of a light chain variable region having an amino acid sequence set forth as SEQ ID NO: 3.

30. The method of claim 29, wherein the CDR1, CDR2, and CDR3 domains of the heavy chain variable region have amino acid sequences set forth as SEQ ID NOs: 4, 5, and 6, respectively; and the CDR1, CDR2, and CDR3 domains of the light chain variable region have amino acid sequences set forth as SEQ ID NOs: 7, 8, and 9, respectively.

31. The method of claim 30, wherein the heavy chain variable region comprises the amino acid sequence set forth in SEQ ID NO: 2, and the light chain variable region comprises the amino acid sequence set forth in SEQ ID NO: 3.

32. The method of claim 28, wherein the anti-NAMPT antibody comprises:
CDR1, CDR2, and CDR3 domains of a heavy chain variable region having an amino acid sequence set forth as SEQ ID NO: 10; and
CDR1, CDR2, and a CDR3 domains of a light chain variable region having an amino acid sequence set forth as SEQ ID NO: 11.

33. The method of claim 32, wherein the CDR1, CDR2, and CDR3 domains of the heavy chain variable region have amino acid sequences set forth as SEQ ID NOs: 12, 13, and 14, respectively; and the CDR1, CDR2, and CDR3 domains of the light chain variable region have amino acid sequences set forth as SEQ ID Nos: 15, 16, and 17, respectively.

34. The method of claim 33, wherein the heavy chain variable region comprises the amino acid sequence set forth in SEQ ID NO: 10, and the light chain variable region comprises the amino acid sequence set forth in SEQ ID NO: 11.

35. The method of claim 28, wherein the anti-NAMPT antibody comprises:
CDR1, CDR2, and CDR3 domains of a heavy chain variable region having an amino acid sequence set forth as SEQ ID NO: 23; and
CDR1, CDR2, and a CDR3 domains of a light chain variable region having an amino acid sequence set forth as SEQ ID NO: 3.

36. The method of claim 35, wherein the CDR1, CDR2, and CDR3 domains of the heavy chain variable region have amino acid sequences set forth as SEQ ID NOs: 4, 24, and 6, respectively; and the CDR1, CDR2, and CDR3 domains of the light chain variable region have amino acid sequences set forth as SEQ ID NOs: 7, 8, and 9, respectively.

37. The method of claim 36, wherein the heavy chain variable region comprises the amino acid sequence set forth in SEQ ID NO: 23, and the light chain variable region comprises the amino acid sequence set forth in SEQ ID NO: 3.

38. The method of any one of claims 1-37, wherein the subject has or is at risk of developing cardiac ischemia.

39. The method of any one of claims 1-37, wherein the subject has or is at risk of developing traumatic brain injury.

40. The method of any one of claims 1-37, wherein the subject has or is at risk of developing cancer.

41. The method of any one of claims 1-37, wherein the subject has or is at risk of developing chorioamnionitis.

42. The method of any one of claims 1-37, wherein the subject has or is at risk of developing NASH.

43. The method of any one of claims 1-37, wherein the subject has or is at risk of developing renal fibrosis.


SEQUENCE LISTING (24 sequences)

SEQ ID NO: 1 - protein, 491 aa, Homo sapiens [sequence omitted]

SEQ ID NO: 2 - protein, 112 aa, Artificial Sequence
  AB 1 heavy chain variable region (VH)
QVQLVQSGAEVTKPGASVKVSCKASGYTFTSYWMQWVRQAPGQGLEWVGEIDPSNSYTNYNQKFRGRVTLTRDTSTTTVYMELSSLRSEDTAVYYCARGGYWGQGTTVTVSS

SEQ ID NO: 3 - protein, 112 aa, Artificial Sequence
  AB 1 and AB 3 light chain variable region (VL)
DIVMTQSPLSLPVTPGEPASISCRSSKSLLHSQGITYLYWYLQKPGQSPQLLIYQLSNRASGVPDRFSGSGSGTDFTLKISRVEAEDVGVYYCVQNLELPYTFGGGTKLEIK

SEQ ID NO: 4 - protein, 10 aa, Artificial Sequence
  AB 1 and AB 3 CDR-H1
GYTFTSYWMQ

SEQ ID NO: 5 - protein, 17 aa, Artificial Sequence
  AB 1 CDR-H2
EIDPSNSYTNYNQKFRG

SEQ ID NO: 6 - protein, 5 aa, Artificial Sequence
  AB 1  and AB 3 CDR-H3
ARGGY

SEQ ID NO: 7 - protein, 16 aa, Artificial Sequence
  AB 1 and AB3 CDR-L1
RSSKSLLHSQGITYLY

SEQ ID NO: 8 - protein, 7 aa, Artificial Sequence
  AB 1 and AB 3 CDR-L2
QLSNRAS

SEQ ID NO: 9 - protein, 9 aa, Artificial Sequence
  AB 1 and AB 3 CDR-L3
VQNLELPYT

SEQ ID NO: 10 - protein, 120 aa, Artificial Sequence
  AB 2 heavy chain variable region (VH)
EVQLVQSGAEVKKPGESLRISCKASGYTFTSYWMHWVRQMPGKGLEWMGEIDPSDSYTNYNQKFKGHVTISADKSISTAYLQWSSLKASDTAMYYCAKSNYVVPWYFDVWGQGTLVTVSS

SEQ ID NO: 11 - protein, 112 aa, Artificial Sequence
  AB 2 light chain variable region (VL)
EIVLTQSPGTLSLSPGERATLSCRSSKSLLHSNGITYLYWYQQKPGQAPRLLIYQMSNLASGIPDRFSGSGSGTDFTLTISRLEPEDFAVYYCAQNLELPWTFGGGTKLEIK

SEQ ID NO: 12 - protein, 10 aa, Artificial Sequence
  AB 2 CDR-H1
GYTFTSYWMH

SEQ ID NO: 13 - protein, 17 aa, Artificial Sequence
  AB 2 CDR-H2
EIDPSDSYTNYNQKFKG

SEQ ID NO: 14 - protein, 13 aa, Artificial Sequence
  AB 2 CDR-H3
AKSNYVVPWYFDV

SEQ ID NO: 15 - protein, 16 aa, Artificial Sequence
  AB 2 CDR-L1
RSSKSLLHSNGITYLY

SEQ ID NO: 16 - protein, 7 aa, Artificial Sequence
  AB 2 CDR-L2
QMSNLAS

SEQ ID NO: 17 - protein, 9 aa, Artificial Sequence
  AB 2 CDR-L3
AQNLELPWT

SEQ ID NO: 18 - DNA, 401 bp, Artificial Sequence
  probe for detecting rs7789066
aatgtgggctttgtttatggtagtatttttttaagatgcaaaatttgatcttgcaatctttgagttgaatttgcagttttaaaataaaaaggtcttatatctgtgcaaagaaaaaatattgtattgacattgcttgttaaattaaggagtgaggcctgcacaagtattagtaatgtgaatcctcacagtagtctccagagaaaaaaaatgacaatgaagtcatgttaccaataggacaatcaccatttgcctgagatagaaataggcacattctctatgtaactacatgcttaagctggagcaattcagaattaattggggtttagaactatgaaattatcactgaaaacagagccaagatttcattttaaaatggcctcccctgaaagacagtttaacag

SEQ ID NO: 19 - DNA, 401 bp, Artificial Sequence
  probe for detecting rs61330082
agtggaacttgtgaattgagattcatagtggaacttgtgaattgagattcatctcgaaactggaggcatggctgagacttctaataaagacaacctcagtcaacactatgtcttgaagtcagtatatatttttgacaatcacctcatctacacgtagatacaatacagggcaaagatcatggaagtggaaggtatcaccaggcactcaccaatgtagtaaatactagtacacttacaattattttcagcaacgaggtttgaaacaagagggcttatgtatttattggttgatcttccctgtgttttaccggggaaaattatttgtaaacgcatttaaacaaattattatttctattttgagacggagtctcgctctgtcgcccaggctggagtgcagtggc

SEQ ID NO: 20 - DNA, 401 bp, Artificial Sequence
  probe for detecting rs9770242
ccgctttcctccggcggctctgtctatggctgagctctttgatcctttgagagatggtttgacttttcccgagcaaagagcctgcgttgaaaagcgggggtggaattcagtcctcacagataatgaggggacaagacctaattgaaccgagtattgccgggaaggaaaaggcaacgggccaagcctttgacagggtgcgacactgacttttatcatcgttatagtctttaaatcctgggaaacgagttggcaaccccaaaataaagaagtgtaatgacgtctgatgacttcacccaaatacagaccattccaagaaagacttgcgcagttctcatgcgtggttgcgtttttgcataaaactaagattccctttgtccgcatgtttaatagcttaaaaataa

SEQ ID NO: 21 - DNA, 401 bp, Artificial Sequence
  probe for detecting rs59744560
gagctgcggtgaggagtgaggctgaggggcccctttcatctgatgcagcgactccgctttcctccggcggctctgtctatggctgagctctttgatcctttgagagatggtttgacttttcccgagcaaagagcctgcgttgaaaagcgggggtggaattcagtcctcacagataatgaggggacaagacctaattgaaccgagtattgccgggaaggaaaaggcaacgggccaagcctttgacagggtgcgacactgacttttatcatcgttatagtctttaaatcctgggaaacgagttggcaaccccaaaataaagaagtgtaatgacgtctgatgacttcacccaaatacagaccattccaagaaagacttgcgcagttctcatgcgtggttgcgtt

SEQ ID NO: 22 - DNA, 401 bp, Artificial Sequence
  probe for detecting rs1319501
gggagctctggcggactccccacctcggttcccccgccttcaccccgtcaccctccgggggccgagaaagggcggggcgcggcagcgcgctgcgcagtgcgcggaggcggggcggggaggaggacgtgatgcacgcgctcttcctcccagacgccagctctgggaagctggaggcagcggggcagccccggcgcgtgacccgggcgcttacctaagttcgagttcccggcacgggcgcgggagggcggggcctggagggggcgttcccagctttgccagtgccacgaggagccggttcgcccgccccgcctgggaccttccgtcctacccagtcctggccggttttctgggtcctcctgaagtcacgccacccggctagggggcgaggagcctcctactgc

SEQ ID NO: 23 - protein, 112 aa, Artificial Sequence
  AB 3 heavy chain variable region (VH)
QVQLVQSGAEVTKPGASVKVSCKASGYTFTSYWMQWVRQAPGQGLEWVGEIEPSNSYTNYNQKFRGRVTLTRDTSTTTVYMELSSLRSEDTAVYYCARGGYWGQGTTVTVSS

SEQ ID NO: 24 - protein, 17 aa, Artificial Sequence
  AB 3 CDR-H2
EIEPSNSYTNYNQKFRG